(19) United States
(12) Patent Application Publication
(10) Pub. No.: US 2026/0274994 A1
(43) Pub. Date: Sep. 17, 2026

(54) COMPOSITIONS FOR ANTIGEN-SPECIFIC IMMUNE CELL ACTIVATION

(71) Applicant: Northwestern University, Evanston, IL (US)

(72) Inventors: Ashima Shukla, Evanston, IL (US); Vipul Shukla, Evanston, IL (US)

(21) Appl. No.: 19/470,769

(22) PCT Filed: Mar. 29, 2024
(86) PCT No.: PCT/US2024/022358

§ 371 Date: Sep. 29, 2025

Related U.S. Application Data
(60) Provisional application No. 63/493,486, filed on Mar. 31, 2023.

(51) Int. Cl. C07K 16/44; A61K 40/11; A61K 40/31; A61K 40/40; A61K 47/68; A61P 35/00; C07K 14/705; C12N 5/0783

(52) U.S. Cl. CPC C07K 16/44; A61K 40/11; A61K 40/31; A61K 40/40; A61K 47/6849; A61P 35/00; C07K 14/7051; C07K 14/70521; C12N 5/0636; C07K 2317/622; C07K 2319/02; C12N 2510/00

(57) ABSTRACT

Disclosed are engineered polynucleotides for expressing chimeric antigen receptors (CARs) that bind a hapten and antibodies conjugated to hapten molecules. Also disclosed are methods for co-administering cells expressing the anti-hapten CARs and the hapten-modified antibody molecule for treating a cancer.

### CROSS-REFERENCE TO RELATED APPLICATIONS

This patent application claims the benefit of, and priority to, U.S. Provisional Patent Application No. 63/493,486, filed Mar. 31, 2023. The content of which is incorporated herein by reference in its entirety.

### REFERENCE TO AN ELECTRONIC SEQUENCE LISTING

The contents of the electronic sequence listing (702581.02492.xml; Size: 35,682 bytes; and Date of Creation: Mar. 29, 2024) is herein incorporated by reference in its entirety.

### BACKGROUND OF THE INVENTION

Chimeric Antigen Receptors (CAR) are synthetically assembled proteins designed by fusion of antigen recognition domains of antibodies to the intracellular signaling domains of the T cell receptor (TCR) complex. Human T cells engineered to express CARs (CAR-T) have revolutionized the outcomes of refractory B cell malignancies, leading to durable clinical responses in ~40% patients. Though CAR-T cells show promise in relapsed/refractory B cell malignancies, several challenges remain for developing CAR-T cells as standard-of care for other hematological malignancies and solid tumors. It is often difficult to identify surface markers that are unique to cancer cells and amenable to CAR-T cell-based targeting, especially in solid tumors. Even in cases where such markers have been identified, counteracting dysfunctional or “exhausted” T cell states in a highly immuno-suppressive tumor microenvironment continues to be a major hurdle. Moreover, hyperactivity of CAR-T cells in patients is shown to be associated with fatal side-effects, such as cytokine release syndrome (CRS). Thus, there are imminent needs to optimize the design and application of current CAR-T cell-based therapeutics.

### SUMMARY OF THE INVENTION

In a first aspect, provided herein is an engineered polynucleotide encoding a chimeric antigen receptor (CAR) comprising an extracellular antigen binding region that binds to a hapten; and an intracellular immune cell activation domain. In embodiments, the hapten is a nitrophenol (NP). In embodiments, the NP is 4-hydroxy-3-nitrophenylacetyl.

In embodiments, the antigen binding region comprises a sequence having at least 95% identity to SEQ ID NO: 2, SEQ ID NO: 3, or SEQ ID NO: 4. In embodiments, the immune cell activation domain comprises at least two of a CD28 signaling domain, a 4-1BB domain, and a CD3ζ signaling domain. In embodiments, the CAR comprises a sequence having at least 95% identity to SEQ ID NO: 1, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 22.

In another aspect, provided herein is a construct comprising any of the engineered polynucleotides described herein operably linked to a heterologous promoter.

In another aspect, provided herein is a chimeric antigen receptor encoded by any of the engineered polynucleotides described herein.

In another aspect, provided herein is a host cell comprising any of the engineered polynucleotides or CARs described herein. In embodiments, the host cell is an immune cell. In embodiments, the host cell is a T cell, a B cell, a natural killer (NK) cell, an invariant natural killer T (iNKT) cell, a macrophage, or an innate lymphoid cell.

In an aspect, provided herein is a modified antibody molecule, wherein the antibody molecule is conjugated to at least one hapten group. In embodiments, the hapten is a nitrophenol. In embodiments, the nitrophenol is 4-hydroxy-3-nitrophenylacetyl. In embodiments, the antibody molecule comprises a binding region specific to a tumor specific antigen or an antigen associated with an autoimmune disease. In embodiments, the tumor specific antigen is CD19 or PD1. In embodiments, the antibody molecule is selected from a monoclonal antibody, a single chain antibody, a nanobody, a T cell engager, and a NK cell engager.

In another aspect, provided herein is a kit comprising any of the engineered polynucleotides, CARs, or host cells described herein; and any of the modified antibody molecule described herein, wherein the CAR antigen binding region binds to the hapten group conjugated to the modified antibody molecule.

In another aspect, provided herein is a method of treating a cancer or an autoimmune disease in a subject, the method comprising administering to the subject a therapeutically effective amount of a first pharmaceutical composition comprising any of the modified antibody molecules described herein; and a pharmaceutically acceptable carrier.

In embodiments, the method further comprises administering to the subject a therapeutically effective amount of a second pharmaceutical composition comprising any of the host cells described herein; and a pharmaceutically acceptable carrier; wherein the CAR antigen binding region binds to the hapten group conjugated to the modified antibody molecule. In embodiments, the second pharmaceutical composition is administered before the first pharmaceutic composition.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Schematic of MASS-CAR strategy. The illustrated MASS-CAR consists of the leader sequence, signal sequence, anti-NP single chain variable fragment, CD28 and/or 41-BB, CD3 zeta, P2A, and Thy1.1 domains. MASS-CAR recognizes NP conjugates to antibodies, nanobodies, ligands-receptor pairs, bi-specific or tri-specific T cell engagers, Natural Killer cell engagers, etc.

FIGS. 2A-2D. Anti-NP CARs are functional and can enhance cytokine production upon activation. Anti-NP CAR T cells and mock (Empty Vector) CAR T cells were incubated with NP-2 BSA at different concentrations. FIG. 2A. Flow cytometry plots showing IFNγ and TNFα positive empty vector and anti-NP CAR transduced CD8 T cells. FIG. 2B. Bar graph quantifying the percent of IFNγ and TNFα positive T cells stimulated for 24 h with different concentrations of carrier protein BSA conjugated with 2 molecules of NP (NP2-BSA). FIG. 2C. Schematic of MASS-CAR variants with different affinity towards NP. FIG. 2D. Bar graph quantifying the IFNγ and TNFα positive T cells stimulated for 24 h with different concentrations carrier protein BSA conjugated with 2 molecules of NP (NP2-BSA). Bars, from left to right, are Mock, NP-Original, W33L, and K59R. **p value>0.001; two-way ANOVA.

FIGS. 3A-3B. NP modification does not alter antibody function. BJAB cell line was treated with PBS, anti-CD19-NP, and anti-CD19 to test if NP-modification can affect the antigen binding. FIG. 3A. Histogram showing the levels of CD19 expression on the surface of BJAB cells when treated with PBS, anti-CD19-NP (5 ug/ml), and anti-CD19 (5 ug/ml) overnight. FIG. 3B. Bar graph for the CD19 expression at different concentrations of anti-CD19-NP and anti-CD19.

FIGS. 4A-4B. Anti-NP CARs can be stimulated with NP-labeled antibodies. Anti-NP CAR T cells were stimulated with anti-CD19-NP and anti-CD19 at different concentrations. FIG. 4A. Bar graph of TNFalpha and Interferon-gamma secreting T cells upon anti-CD19-NP and anti-CD19 stimulation at different concentrations. The bars, from left to right are CD19-NP 1 μg/ml, CD19-NP 0.2 ug/ml, CD19 1 μg/ml, CD19 0.2 ug/ml, and PBS. FIG. 4B. FACs plots showing cytokine production in Mock CAR-T cells and anti-NP CAR T cells.

FIGS. 5A-5C. MASS-CAR is switchable and is efficient in killing cancer cells in vitro. B16F10 melanoma cells express PDL-1 at high levels (data not shown). Moreover, we stably transduced B16F10 cells to express human CD19 to compare the efficacy with current CAR-T therapy. Using these B16F10 cells as a model we tested the cytotoxicity of MASS-CAR in vitro. FIG. 5A. Schematic of MASS-CAR using two different NP-labelled antibodies to target melanoma cells. FIG. 5B. Bar graph quantifying the cytotoxicity of MASS-CAR in comparison with Mock T cells in the presence of NP-labeled anti-mouse PDL1, unlabeled anti-PDL1, and isotype control antibodies. FIG. 5C. Bar graph quantifying the cytotoxicity of MASS-CAR in comparison with Mock T cells and conventional CD19-CAR in the presence of NP-labeled anti-human CD19 and unlabeled anti-human CD19. ***p-value>0.001; **** p-value>0.0001; two-way ANOVA on graph pad prism.

FIGS. 6A-6C. MASS-CAR efficiently targets tumor cells in vivo. FIG. 6A. Schematic of timeline for in vivo MASS-CAR testing with engager. FIGS. B and C. Line graph of individual (FIG. 5B) and group average (FIG. 5C) of tumor volume (volume=[(length×width²)/2] of tumors from mice measured every other day. Mice receiving T cells transduced with empty lentiviral vector are called Mock; Mice receiving T cells transduced with mouse version of conventional CD19-CAR are called CD19-CAR; Mice receiving MASS-CAR with unlabeled antibody are called MASS-CAR+Un-Engager; Mice receiving MASS-CAR with NP-labeled antibody are called MASS-CAR+Lb-Engager. Two-way ANOVA multiple comparison on graph pad prism.

FIGS. 7A-7D. MASS-CARs get recruited at the tumors in presence NP-labelled engagers (Lb-Engager). FIG. 7A. Flow plots showing Thy1.1 positive cells in tumors. FIG. 7B. Bar graph showing the percentage of transferred CAR-T cells in mice tumors. FIG. 7C. Flow plot of showing Tim3 and PD-1 double positive cells. FIG. 7D. Bar graph showing the percentage of exhausted T cells in tumors stained with Tim3 and PD-1. Unpaired T-Test on graphpad prism.

FIGS. 8A-8E. MASS-CARs with labelled engagers enhances the recruitment of endogenous immune cells. Mice with similar size of tumors were sacrificed on Day 19, tumors and draining lymph nodes were isolated. Tumors were homogenized and immunophenotyped by flow cytometry. FIG. 8A. Bar graph displaying the frequencies of endogenous immune cells, CD45.2 positive and CD45.1 negative cells gated on single cells. FIG. 8B. Bar graph displaying the frequency of Natural Killer cells, DX-5 positive cells in CD45.2 positive gated cells. FIG. 8C. Bar graph displaying myeloid derived suppressor cells (MDSCs), Ly6G positive cells in CD11b positive gated from CD45.2 positive gated cells. FIG. 8D. Bar graph displaying the frequency of B220+CD19+ B cells in CD45.2 positive gated cells. FIG. 8E. Ly6C positive cells in CD11b positive gated from CD45.2 positive gated cells. One-way ANOVA on graph pad prism.

FIGS. 9A-9C. MASS-CARs with labelled engagers enhances the recruitment of endogenous immune cells. Mice with similar size of tumors were sacrificed on Day 19, tumors and draining lymph nodes were isolated. Lymph nodes were homogenized and immunophenotyped by flow cytometry. FIG. 9A. Bar graph displaying frequencies CD45.1.2 positive CAR-T cells in the inguinal lymph nodes. FIG. 9B. Bar graph displaying frequencies of CD62L CD44 double positive central memory (T CM) CAR-T cells. FIG. 9C. Bar graph showing the frequencies of CD62L+CD44− stem cell memory (TSCM) CAR-T cells. One-way ANOVA on graph pad prism.

FIG. 10. Anti-CD19 antibody-hapten conjugate is more efficacious than anti-CD19 antibody. Human CD19 expressing B16F10 melanoma cells (50,000 per mouse) were subcutaneously inoculated in mice. Day 13 mice were given 50 ug/mouse of antibody every other day for 5 doses. The displayed graph shows the average tumor volume of group treated with anti-CD19, anti-CD19-NP and untreated mice. *p-value >0.01; ****p-value >0.0001; two-way ANOVA.

### DETAILED DESCRIPTION OF THE INVENTION

The idea to arm the body's own immune system to eliminate precarious cancer cells, known as cancer immunotherapy, has completely transformed our thinking of anti-cancer treatments. However, at present, cancer immunotherapies are not widely applicable to all cancer types and there continues to be several unmet needs to design immunotherapies that are robust, effective, and easily generalizable to multiple cancers. Described herein are compositions and methods to overcome these challenges comprising MASS-CAR (Modified Antibody Specific Switchable CAR), a strategy which uses chemical labeling of antibodies to direct CAR-T cell activity to specific tumors while also leveraging the therapeutic benefits of antibody biologics, leading to more holistic anti-tumor immune responses. The present disclosure provides for the development of universal CARS that can be used in combination with any modified antibody molecule of choice allowing for the switching of CAR-T cells to different tumor targets.

### Compositions

In a first aspect, disclosed herein is an engineered polynucleotide encoding a chimeric antigen receptor (CAR) comprising an extracellular antigen binding region that binds to a hapten, and an intracellular immune cell activation domain. The hapten may be a nitrophenol (NP). The CAR is configured to bind to a hapten conjugated to a modified antibody molecule. Together, the CAR and the modified antibody molecule can induce an immune response against a cancer cell expressing an antigen targeted by the modified antibody molecule.

The terms “engineered polynucleotide”, “recombinant polynucleotide”, “genetically engineered polynucleotide”, and “genetically modified polynucleotide” refer to any manipulation of a polynucleotide that results in a detectable change in a naturally occurring polynucleotide, wherein the manipulation includes, but is not limited to, changes in the sequence of the polynucleotide or inclusion of non-naturally occurring nucleotides or nucleosides.

As used herein, “a polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, including DNA, RNA, ORFs, analogs and fragments thereof. The polynucleotides disclosed herein may be optimized, for example codon optimized or host cell optimized. For example, the polynucleotide may encode a fusion protein comprising a chimeric antigen receptor. As used herein a “fusion protein” refers to proteins created through the joining of two or more genes that originally coded for separate proteins.

The terms “chimeric antigen receptor”, “chimeric receptor”, and “CAR” refer to a polypeptide having a binding specificity to a desired target and operably connected to (e.g., as a fusion or as separate chains linked by one or more disulfide bonds, etc.) the intracellular part of an immune cell activation domain. When expressed, a CAR is present at the plasma membrane of an immune cell. More particularly, CARs are engineered receptors which, when expressed, graft an antigen specificity onto a cytotoxic cell, such as a T cell, a natural killer (NK) cell, a macrophage, etc. For example, CARs are engineered to give T cells the new ability to target a specific protein. CARs comprise an extracellular domain having at least one antigen specific targeting region that binds to a hapten, a transmembrane domain (TM), and an intracellular domain (ID) including a T cell activation domain, the T cell activation domain comprising one or more co-stimulatory domains (CSD), in a combination that is not naturally found together on a single protein. The antigen specific targeting region may be a single chain antibody (scFv). CARs known in the art typically bind to antigens expressed on tumor cells. The CARs of the present invention bind to a hapten.

The engineered CAR polynucleotide disclosed herein may comprise all elements necessary for the desired expression and function of the CAR. For example, the CAR may include a leader sequence and/or signal peptide for directing expression of the CAR at the cell membrane. The CAR may comprise a transmembrane domain. The CAR may comprise a hinge region linking the extracellular domain to the transmembrane domain. The intracellular domain may comprise one or two cytoplasmic costimulatory domains that enhance T cell activation. Costimulatory domains that may be included in the CARs described herein include, but are not limited, to CD28, CD3ζ, CD27, CD134, and 4-1BB/CD137 domains. CARs may further include factors that enhance T cell expansion, persistence, and anti-tumoral activity, such as cytokine or cytokine signaling domains including, but not limited to, IL-2, IL-12, JAK, and STAT3/5. In exemplary embodiments, the CAR comprises at least two of a CD28 costimulatory domain, a 4-1BB costimulatory domain, and a CD3ζ costimulatory domain. The CD28 costimulatory domain may comprise a sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO: 17. The CD3ζ costimulatory domain may comprise a sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO: 16. The 4-1BB costimulatory domain may comprise a sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO: 18.

A “hapten” is a small molecule that elicits an immune response only when attached to a large carrier, such as a protein. Nitrophenols (NPs) are a known class of small molecule haptens that are immunogenic when conjugated to a carrier. The nitrophenol may be a di-nitrophenol, a tri-nitrophenol, or any other nitrophenol. In exemplary embodiments, nitrophenol is 4-hydroxy-3-nitrophenylacetyl. The hapten may be digoxigenin. The engineered polynucleotide antigen binding region may comprise a sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO: 2 (NP binding domain), SEQ ID NO: 3 (NP binding domain with a W33L mutation), or SEQ ID NO: 4 (NP binding domain with a K59R mutation).

In exemplary embodiments, the CAR comprises a sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO: 1 (NP CAR with CD3ζ and CD28 signaling domains), SEQ ID NO: 5 (NP CAR with CD3ζ and CD28 signaling domains; W33L NP domain mutant), SEQ ID NO: 6 (NP CAR with CD3ζ and CD28 signaling domains; K59R NP domain mutant), SEQ ID NO: 7 (NP CAR with CD3ζ and 4-1BB signaling domains), SEQ ID NO: 8 (NP CAR with CD3ζ and 4-1BB signaling domains; W33L NP domain mutant), SEQ ID NO: 9 (NP CAR with CD3ζ and 4-1BB signaling domains; K59R NP domain mutant), SEQ ID NO: 10 (NP CAR with CD3ζ, 4-1BB and CD28 signaling domains), SEQ ID NO: 11 (NP CAR with CD3ζ, 4-1BB and CD28 signaling domains; W33L NP domain mutant), SEQ ID NO: 12 (NP CAR with CD3ζ, 4-1BB and CD28 signaling domains; K59R NP domain mutant), or SEQ ID NO: 22 (NP CAR with CD3ζ, 4-1BB and CD28 signaling domains; K59R NP domain mutant).

“Percentage of sequence identity” or “percent similarity” is determined by comparing two optimally aligned sequences over a comparison window, wherein the portion of the polynucleotide or peptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “substantial similarity” of polynucleotide or peptide sequences means that a polynucleotide or peptide comprises a sequence that has at least 75% sequence identity. Alternatively, percent identity can be any integer from 75% to 100%. More preferred embodiments include at least: 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

Also disclosed herein is a construct comprising the engineered CAR polynucleotide operably linked to a heterologous promoter.

As used herein, the terms “heterologous promoter,” “promoter,” “promoter region,” and “promoter sequence” refer generally to transcriptional regulatory regions of a gene, which may be found at the 5′ or 3′ side of a polynucleotides described herein, or within the coding region of said polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

The CAR construct described herein may further comprise any component of a T cell receptor (TCR) complex, and/or a TCR associated accessory protein.

The CAR construct may be comprised in a vector. The term “vector” refers to a nucleic acid molecule capable of propagating a nucleic acid segment within it, e.g. the engineered CAR polynucleotide described herein. The term includes the vector as a self-replicating nucleic acid structure as well as a vector incorporated into the genome of a host cell into which it has been introduced. The term vector encompasses “plasmids”, the most commonly used form of vector. Plasmids are circular double-stranded DNA loops into which additional DNA segments (e.g., those encoding peptides) may be ligated. In some embodiments, the vector is a mini-circle DNA (mcDNA) vector. Mini-circle DNA vectors are episomal DNA vectors that are produced as circular expression cassettes devoid of any bacterial plasmid DNA backbone. See, for example, System Biosciences, Mountain View CA, MN501A-1. Their smaller molecular size enables more efficient transfections and offers sustained expression over a period of weeks as compared to standard plasmid vectors that only work for a few days. The vectors may further a comprise heterologous nucleic acid backbone sequence. As used herein, “heterologous nucleic acid sequence” refers to a non-human nucleic acid sequence, for example, a bacterial, viral, or other non-human nucleic acid sequence that is not naturally found in a human. Heterologous backbone sequences may be necessary for propagation of the nucleic acid segment and/or expression of encoded peptides.

A chimeric antigen receptor encoded by the engineered polynucleotide disclosed herein is a provided. As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids. A protein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine).

The vector used herein may be a viral vector. The vector may be a lenti-, baculo-, or adeno-viral/adeno-associated viral vector, preferably a retroviral or lentiviral vector. In exemplary embodiments, the vector is a lentiviral vector.

The CAR constructs and vectors disclosed herein may be expressed in a host cell. As used herein, a “host cell” or “engineered host cell” is the cell in which expresses the engineered polynucleotide or polypeptide. The host cell may be a mammalian cell. The host cell may be a human cell. In preferred embodiments, the host cell is an immune cell. The host cell may comprise a T cell, a natural killer (NK) cell, a B cell, an invariant natural killer T (iNKT) cell, a macrophage, or an innate lymphoid cell. Accordingly, the host cell comprising the CAR protein and/or the engineered polynucleotide may be a CAR-T cell, CAR-NK cell, CAR-B cell, a CAR-macrophage, or a CAR-iNKT cell. The host cell may be derived from a subject, engineered to express the CAR described herein, then used to treat the subject. Any suitable means for delivering the CAR constructs and vectors to the host cell may be used including, but not limited to, transfection, transduction, transformation, and nanoparticle delivery.

A pharmaceutical composition comprising the host cell is provided herein. The pharmaceutical composition comprises the host cell comprising the engineered CAR polynucleotide or protein described herein and a pharmaceutically acceptable delivery vehicle.

As used herein, the term “pharmaceutical composition” refers to a chemical or biological composition suitable for administration to a mammal. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions. Examples of compositions appropriate for such therapeutic applications include preparations for parenteral, subcutaneous, transdermal, intradermal, intramuscular, intracoronarial, intramyocardial, intraperitoneal, intravenous or intraarterial (e.g., injectable), or intratracheal administration, such as sterile suspensions, emulsions, and aerosols. In some cases, pharmaceutical compositions appropriate for therapeutic applications may be in admixture with one or more pharmaceutically acceptable excipients, diluents, or carriers such as sterile water, physiological saline, glucose or the like.

In a second aspect, provided herein is an antibody modified molecule labeled with a hapten. The antibody molecule may be labeled with a nitrophenol (NP). The nitrophenol may be a di-nitrophenol, a tri-nitrophenol, or any other nitrophenol. The antibody may be conjugated with at least one 4-hydroxy-3-nitrophenylacetyl group. The antibody molecule may be modified with the hapten by lysine conjugation, cysteine conjugation or any other suitable labeling techniques. (See, e.g., Stephen J. Walsh et al. Chem Soc Rev 2021, 50, 1305-1353). The hapten-modified antibody molecule may be of any specificity, and antibodies of more than one specificity, such as bispecific antibodies, may be modified with haptens for use as described herein. The hapten-modified antibody molecules may be specific to a tumor specific antigen. Tumor specific antigens include, but are not limited to, HER2, BCMA, CD33, CD123, PD1, PDL1, Tim3, Tim1, LAG3, TIGIT, TNFRSF17, SDC1, MS4A1, CD22, TNFRSF8, CD33, CD38, CDS, NCAM1, CD70, ULBP1, ULBP2, IL1RAP, CEACAMS, MET, EPHA2, ERBB2, GPC3, Muc1, PDCD1, CD274, KDR, IL13RA2, FOLH1, FAP, CA9, FOLR1, LICAM, ROR1, CD23, CD44, CD174, SLAMF7, GD2, PSCA, GPNMB, CD276, CSPG4, CD133, TEMIPSA, TRP-2, EpCAM, GPC3, MSLN, EGFR, EGFRVIII, p21,RAS, p53, CDK-4, β-catenin, Notch receptors, and MAGE. In exemplary embodiments, the antibody antigen binding region is specific to CD19 or PD1. The hapten-modified antibody molecules may be specific to an antigen associated with an autoimmune disease. Antigens associated with autoimmune diseases include, but are not limited to aminoacyl tRNA synthetase, Mi-2, signal recognition particle (SRP), transcriptional intermediary factor 1-gamma (TIF1-γ; TRIM 33), nuclear matrix protein-2 (NXP2; MORC3), 3-hydroxy-3-methylgultaryl-coA reductase (HMGCR), melanoma-associated differentiation gene-5 (MDA5), small ubiquitin-like modifier activating enzymes SAE-1 and SAE-2, topoisomerase-1, centromere proteins A, B & C (“CENPs”), fibrillarin, nucleophosmin (NPM; B23), RNA polymerases I, II & III, EXOSC9, EXOSC10, Ku 70/80 components of DNA-dependent protein kinase, Ro52 (TRIM 21), Ro60, La (SS-B), gamma interferon-inducible protein-16 (IFI16), proteinase-3 (PR3), myeloperoxidase (MPO), a citrullinated protein, peptidyl arginine-deiminase-4 (PAD4), double stranded DNA (dsDNA), a component of the Sm splicing ribonucleoprotein, U1-RNP, ribosomal protein P, cardiolipin, an anionic phospholipid/protein complex, N-methyl-D-aspartate (NMDA) receptor, and tissue transglutaminase.

The term “antibody molecule” as used herein refers to immunoglobulin molecules or other molecules which comprise an antigen binding domain. The term “antibody molecule” as used herein is thus intended to include whole antibodies (e.g., IgG, IgA, IgE, IgM, or IgD), monoclonal antibodies, chimeric antibodies, humanized antibodies, and antibody fragments, including single chain variable fragments (ScFv), single domain antibodies (nanobodies), and antigen-binding fragments, genetically engineered antibodies, among others, as long as the characteristic properties (e.g., ability to bind CD30) are retained. The term “antibody fragment” as used herein is intended to include any appropriate antibody fragment that displays antigen binding function, for example, Fab, Fab′, F(ab′)2, scFv, Fv, dsFv, ds-scFv, Fd, mini bodies, monobodies, and multimers thereof and bispecific antibody fragments.

As stated above, antibody fragments/antigen binding fragments comprise an antigen binding domain. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (e.g. single chain antibodies or single chain Fv (scFv), (see for instance Bird et al., Science 242, 423-426 (1988) and Huston et al., PNAS USA 85, 5879-5883 (1988)).

The term “fragment” as used herein refers to fragments of biological relevance (functional fragment), e.g., fragments which can contribute to or enable antigen binding, e.g., form part or all of the antigen binding site or can contribute to the prevention of the antigen interacting with its natural ligands. Fragments may comprise a heavy chain variable region (V_{H} domain) and light chain variable region (V_{L}). Fragments may comprise one or more of the heavy chain complementarity determining regions (CDRHs) of the antibodies or of the V_{H} domains, and one or more of the light chain complementarity determining regions (CDRLs), or V_{L} domains to form the antigen binding site.

Antibody molecules can be genetically engineered from CDRs and monoclonal antibody sequences by using conventional techniques such as, for example, synthesis by recombinant techniques or chemical synthesis. Techniques for producing antibody fragments are well known and described in the art.

One may wish to engraft one or more CDRs from monoclonal antibodies into alternate scaffolds. For example, standard molecular biological techniques can be used to transfer the DNA sequences encoding the antibody's CDR(s) to (1) full IgG scaffold of human or other species; (2) a scFv scaffold of human or other species, or (3) other specialty vectors. If the CDR(s) have been transferred to a new scaffold all of the previous modifications described can also be performed. For example, one could consult Biotechnol Genet Eng Rev, 2013, 29:175-86 for a review of useful methods.

The antibody molecules can be wholly or partially synthetically produced. Thus, the antibody molecule may be from any appropriate source, for example recombinant sources and/or produced in transgenic animals or transgenic plants. Thus, the antibody molecules can be produced in vitro or in vivo. The antibody molecules may comprises all or a portion of a heavy chain constant region, such as an IgG1, IgG2, IgG3, IgG4, IgA1, IgA2, IgE, IgM or IgD constant region.

The antibody molecules may comprise all or a portion of a kappa light chain constant region or a lambda light chain constant region. All or part of such constant regions may be produced wholly or partially synthetic. Appropriate sequences for such constant regions are well known and documented in the art.

The term “complementarity determining regions” or “CDRs,” as used herein, refers to part of the variable chains in immunoglobulins (antibodies) and T cell receptors, generated by B-cells and T-cells respectively, where these molecules bind to their specific antigen. As the most variable parts of the molecules, CDRs are crucial to the diversity of antigen specificities generated by lymphocytes. There are three CDRs (CDR1, CDR2 and CDR3), arranged non-consecutively, on the amino acid sequence of a variable domain of an antigen binding site. Since the antigen binding sites are typically composed of two variable domains (on two different polypeptide chains, heavy and light chain), there are six CDRs for each antigen binding site that can collectively come into contact with the antigen. A single whole antibody molecule has two antigen binding sites and therefore contains twelve CDRs. Sixty CDRs can be found on a pentameric IgM molecule.

Within the variable domain, CDR1 and CDR2 may be found in the variable (V) region of a polypeptide chain, and CDR3 includes some of V, and all of diversity (D, heavy chains only) and joining (J) regions. Since most sequence variation associated with immunoglobulins and T cell receptors is found in the CDRs, these regions are sometimes referred to as hypervariable regions. Among these, CDR3 shows the greatest variability as it is encoded by a recombination of VJ in the case of a light chain region and VDJ in the case of heavy chain regions. The tertiary structure of an antibody is important to analyze and design new antibodies. The human V_{H} complex is composed of approximately 100 gene segments per haploid genome, including at least 51 functional genes, as judged by successful rearrangement in cloned cDNA. On the basis of nucleic acid sequence homology, the V_{H} genes have been grouped into 6-7 families (VH 1-7). Among the seven families, the VH3 family is the largest. The antibody molecules disclosed herein may be derived from the VH3-74 family or the VH3-33 family or its paralog.

The terms “monoclonal antibody” or “monoclonal antibody composition” as used herein refer to a preparation of antibody molecules of a single amino acid composition that specifically binds to a single epitope of the antigen.

The term “chimeric antibody” refers to an antibody comprising a variable region, i.e., binding region, from one source or species and at least a portion of a constant region derived from a different source or species, usually prepared by recombinant DNA techniques. Other forms of “chimeric antibodies” are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. In some embodiments, the antibodies are chimeric antibodies including heavy chain constant domains from non-human mammals (e.g., mouse, rat, rabbit, or non-human primate). In some embodiments, the antibodies disclosed in the present invention are chimeric antibodies including constant regions from rabbit heavy chain immunoglobulin sequences. Suitable heavy chain constant region sequences from non-human mammals, including mouse, rat, rabbit, and non-human primate are known in the art.

The antibody molecules disclosed in the present invention may be human antibody molecules, as they may include the constant region from human germline immunoglobulin sequences. The term “recombinant human antibody” includes all human antibodies that are prepared, expressed, created, or isolated by recombinant means, such as antibodies isolated from a host cell such as an SP2-0, NS0 or CHO cell (like CHO K1) or from an animal (e.g., a mouse) that is transgenic for human immunoglobulin genes or antibodies expressed using a recombinant expression vector transfected into a host cell. Such recombinant human antibodies have variable and, in some embodiments, constant regions derived from human germline immunoglobulin sequences in a rearranged form.

An antibody molecule may also be T cell engager or a natural killer (NK) cell engager. A “T cell engager” refers to an antibody engineered to bind a target antigen expressed on a cancer cell and at least one molecule on a T cell, such as CD3. As such, T cell engagers are multispecific. The terms “bispecific T cell engager”, “BiTE”, and “BTE” are used interchangeable herein and refer to the antibody molecules that are capable of binding two distinct antigens at the same time. Particularly, the bispecific antibodies are capable of binding to the surface of tumor cells and T cells simultaneously, allowing for activation of the T cells and the targeting killing of tumor cells bound to the bispecific antibody.

The T cell engager may be tri-specific. The term “tri-specific” means that the binding protein is able to specifically bind to at least three distinct moieties (e.g., antigen binding sites). Typically, a tri-specific molecule comprises three different binding sites, each of which is specific for a different moiety (e.g., antigen). The tri-specific binding proteins described herein may be capable of simultaneously binding three moieties, particularly three moieties expressed on two distinct cells (e.g., a tumor cell and a T cell). The tri-specific T cell engagers may be capable of binding two cancer cell antigens and a T cell antigen. The terms “trispecific T cell engager”, “TriTE”, and “TrTE” are used interchangeable herein and refer to the antibody molecules that are capable of binding three distinct antigens at the same time. Particularly, the tri-specific antibodies are capable of binding to the surface of tumor cells and T cells simultaneously, allowing for activation of the T cells and the targeting killing of tumor cells bound to the tri-specific antibodies.

A pharmaceutical composition comprising the hapten-modified antibody molecule is provided. The composition comprises the antibody and a pharmaceutically acceptable delivery vehicle.

In a third aspect, provided herein is a kit comprising an engineered CAR polynucleotide, CAR protein, or engineered host cell described herein; and a hapten-modified antibody molecule described herein, wherein the CAR antigen binding region binds to the hapten group conjugated to the modified antibody molecule. The engineered host cell and hapten-modified antibody are able to functionally interact with each other in such a way that when the modified antibody binds to the host cell and a cancer cell, the host cell exerts an immune response upon the cancer cell. Activation of the immune response may include T cell activation and response, including cytotoxicity, as well as a B cell or antibody mediated response. Activation of the immune response in response to the compositions and methods provided herein may comprise an increase in IFNγ, TNFα, perforins and granzyme B, activation and proliferation of anti-hapten CAR CD8+ T cells. Preferably, the engineered CAR polynucleotide/CAR protein/engineered host cell and the hapten-modified antibody are provided in separate containers.

### Methods

In a fourth aspect, provided herein is a method of treating a cancer or an autoimmune disease, or inducing an immune response in a subject, the method comprising administering to the subject a therapeutically effective amount of a pharmaceutical composition comprising a hapten-modified antibody molecule described herein.

Cancer is a group of diseases involving abnormal cell growth with the potential to invade or spread to other parts of the body. Treating a cancer can be characterized by at least one of the following: (a) reducing, slowing or inhibiting growth of cancer and cancer cells, including slowing or inhibiting the growth of metastatic cancer cells; (b) preventing further growth of tumors; (c) reducing or preventing metastasis of cancer cells within a subject; (d) reducing or ameliorating at least one symptom of cancer; and (e) extending the survival of the subject. Treating cancer in a subject also includes the reduction of the number of tumor cells within the subject. When the engineered host cell is an immune cell, the term “treatment” may further include activating an immune response in the subject, wherein the immune response targets the cancer cells. The cancer may be a solid tumor or a hematological malignancy. Cancers that may be treated using the compositions and methods described herein include, but are not limited to B cell cancers, myeloid leukemias, T cell cancers, breast cancers, pancreatic cancers, colorectal cancers, brain cancers, head and neck cancers, melanomas, etc.

Autoimmune diseases are a group of diseases arising from when the body's immune system mistakenly attacks healthy tissues, organs, and cells. In methods for treating autoimmune diseases, the hapten-modified antibody molecule may be specific to an antigen associated with an autoimmune disease. Treating an autoimmune disease can be characterized by reducing are preventing one or more symptoms caused by an autoimmune disease. Symptoms include, but are not limited to fatigue, frequent fevers, joint pain and swelling, stomach pain or digestion issues, and swollen glands. Autoimmune diseases that may be treated using the compositions and methods described herein include, but are not limited to from scleroderma, Sjogren's syndrome, vasculitis, rheumatoid arthritis (RA), systemic lupus erythematosus (SLE), and celiac disease. For further information, see Rosen and Casciola-Rosen “Autoantigens as Partners in Initiation and Propagation of Autoimmune Rheumatic Diseases” Ann. Rev. Immunol. 2016 May 20; 34:395-420, which is incorporated herein by reference.

The method may further comprise administering to the subject a therapeutically effective amount of the engineered host cell described herein, wherein the CAR antigen binding region binds to the hapten group conjugated to the modified antibody. The pharmaceutical composition comprising the host cell may be administered first, followed by the pharmaceutical composition comprising the modified antibody.

An “effective amount” or a “therapeutically effective amount” means the amount of the host cell or modified antibody sufficient to treat the cancer. The amount of the pharmaceutical compositions to be administered is dependent on a variety of factors, including the severity of the condition, the age, sex, and weight of the subject, the frequency of administration, the duration of treatment, and the like. The pharmaceutical compositions may be administered at any suitable dosage, frequency, and for any suitable duration necessary to achieve the desired therapeutic effect. Either or both of the pharmaceutical composition may be administered once per day or multiple times per day, e.g. twice per day or three or more times per day. Either or both of the composition may be administered daily, every other day, every three days, every four days, every five days, every six days, once per week, once every two weeks, or less than once every two weeks. The compositions may be administered for any suitable duration to achieve the desired therapeutic effect, i.e., enhanced transplant tolerance. For example, either or both of the compositions may be administered to the subject for one day, two days, three days, four days, five days, six days, seven days, eight days, nine days, ten days, eleven days, twelve days, thirteen days, two weeks, one month, two months, three months, six months, 1 year, or more than 1 year. Any suitable dose of the pharmaceutical compositions may be used. Suitable doses will depend on the therapeutic agent, intended therapeutic effect, body weight of the individual, age of the individual, and the like. In general, suitable dosages of the disclosed nanocarriers or pharmaceutical compositions comprising the same may range from about 0.025 mg nanocarrier/kg body weight to 200 mg nanocarrier/kg body weight. For example, suitable dosages may be about 0.025 mg/kg, about 0.03 mg/kg, about 0.05 mg/kg, about 0.10 mg/kg, about 0.15 mg/kg, about 0.30 mg/kg, about 0.5 mg/kg, about 0.75 mg/kg, about 1.0 mg/kg, about 1.25 mg/kg, about 1.5 mg/kg, about 1.75 mg/kg, about 2.0 mg/kg, about 3 mg/kg, about 5 mg/kg, about 10 mg/kg, about 25 mg/kg, about 50 mg/kg, about 75 mg/kg, about 100 mg/kg, about 125 mg/kg, about 150 mg/kg, about 175 mg/kg, or about 200 mg/kg.

Suitable regimes for initial administration and further doses or for sequential administrations also are variable, may include an initial administration followed by subsequent administrations, but nonetheless, may be ascertained by the skilled artisan from this disclosure, the documents cited herein, and the knowledge in the art.

Data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any agent of the present technology used in the methods described herein, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As used herein, the term “administering” refers to dispensing or delivering the therapeutic to the subject by any suitable route for delivery of the therapeutic agent to the desired location in the subject, including delivery by either the parenteral or oral route, intramuscular injection, subcutaneous/intradermal injection, intravenous injection, intrathecal administration, buccal administration, transdermal delivery, and administration by the intranasal or respiratory tract route.

The immune response generated by the engineered host cell and hapten-modified antibody can be increased or decreased if changes to the administration of either composition is altered. For example, an immune response will be generated if the engineered cell and the hapten-modified antibody molecule interact, however if administration of the hapten-modified antibody molecule is stopped, the immune response will be less activated. Alternatively, if administration of the engineered host cell is stopped, the host cell-specific immune response will decrease.

Formulations may be designed or intended for oral, rectal, nasal, systemic, transmucosal (including buccal, sublingual, ocular, vaginal and rectal), or parenteral (including subcutaneous, intramuscular, intravenous, intraarterial, intradermal, intraperitoneal, intrathecal, intraocular and epidural) administration. In general, aqueous and non-aqueous liquid or cream formulations are delivered by a parenteral, oral or topical route. In other embodiments, the compositions may be present as an aqueous or a non-aqueous liquid formulation or a solid formulation suitable for administration by any route, e.g., oral, topical, buccal, sublingual, parenteral, aerosol, a depot such as a subcutaneous depot or an intraperitoneal or intramuscular depot. In some cases, pharmaceutical compositions are lyophilized. In other cases, pharmaceutical compositions as provided herein contain auxiliary substances such as wetting or emulsifying agents, pH buffering agents, gelling or viscosity enhancing additives, preservatives, flavoring agents, colors, and the like, depending upon the route of administration and the preparation desired. The pharmaceutical compositions may be formulated according to conventional pharmaceutical practice (see, e.g., Remington: The Science and Practice of Pharmacy, 20^{th} edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York). For intravenous administration, suitable carriers include physiological saline, bacteriostatic water, Cremophor EL® (BASF, Parsippany, N.J., USA) or phosphate buffered saline (PBS).

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity, such as sodium chloride or dextrose. The pH can be adjusted with acids or bases, such as hydrochloric acid or sodium hydroxide. The preparation can be enclosed in ampoules, disposable syringes or multiple-dose vials made of glass or plastic. For convenience of the patient or treating physician, the dosing formulation can be provided in a kit containing all necessary equipment (e.g., vials of drug, vials of diluent, syringes and needles) for a course of treatment. In all cases, a composition for parenteral administration must be sterile and should be formulated for ease of injectability. The composition should be stable under the conditions of manufacture and storage, and must be shielded from contamination by microorganisms such as bacteria and fungi.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

The preferred route may vary with, for example, the subject's pathological condition or age or the subject's response to therapy or that is appropriate to the circumstances. The formulations can also be administered by two or more routes, where the delivery methods are essentially simultaneous or they may be essentially sequential with little or no temporal overlap in the times at which the composition is administered to the subject.

The term “subject” or “patient” are used herein interchangeably to refer to a mammal, preferably a human, to be treated by the methods and compositions described herein. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Preferably, the subject is a human.

In embodiments, the method of treatment may further comprise administering another anti-cancer treatment. Cancer treatment includes, but is not limited to chemotherapy, radiation, bone marrow transplant, surgery and immunotherapy.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are illustrative and should not be interpreted to limit the scope of the claimed subject matter.

### Example 1—MASS-CAR: a Novel Strategy for Cancer Immunotherapy

Chimeric Antigen Receptors (CAR) are synthetically assembled proteins designed by fusion of antigen recognition domains of antibodies to the intracellular signaling domains of the T cell receptor (TCR) complex^{1,2,7}. Human T cells engineered to express CARs (CAR-T cells) have revolutionized the outcomes of refractory B cell malignancies, leading to durable clinical responses in ~40% patients²⁻⁶. Though CAR-T cells show promise in relapsed/refractory B cell malignancies, several challenges remain for developing CAR-T cells as standard-of-care for other hematological malignancies and solid tumors. It is often difficult to identify surface markers that are unique to cancer cells and amenable to CAR-T cell-based targeting, especially in solid tumors. Even in cases where such markers have been identified, counteracting dysfunctional or “exhausted” T cell states in a highly immuno-suppressive tumor microenvironment continues to be a major hurdle². Moreover, hyperactivity of CAR-T cells in patients is shown to be associated with fatal side-effects, such as cytokine release syndrome (CRS)². Thus, there are imminent needs to optimize the design and application of current CAR-T cell-based therapeutics.

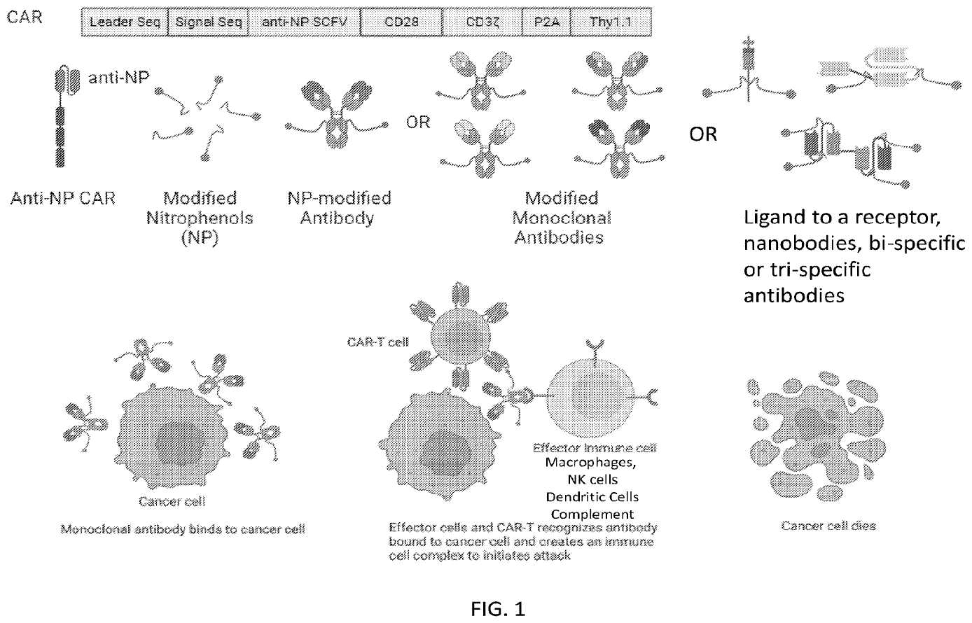

To overcome some of the major challenges for CAR-T cell approaches, we developed a novel strategy that we term, MASS-CAR, for Modified Antibody Specific Switchable CAR (FIG. 1). MASS-CAR uses chemical labeling of antibodies to direct CAR-T cell activity to specific tumor cells, while concurrently engaging multiple components of the adaptive and innate immune systems, which in principle, would promote more holistic and potentially superior anti-tumor responses. Towards this, we generated a novel CAR by fusion of the single chain variable fragments (SCFVs) of a small molecule 4-hydroxy-3-nitrophenacetyl (NP) specific antibody to the CD28 and CD3ζ signaling domains of the TCR (FIG. 1). MASS-CAR employs one or more NP-labelled antibodies to direct CAR-T cell activity in tumors. Moreover, this strategy allows simple toggling of CAR-T cells between on and off states relying on the administration of NP-labelled antibodies, translating to fewer side-effects associated with CAR-T cell hyperactivity. Altogether, the MASS-CAR is a highly versatile and modular strategy that can allow targeting of multiple tumor markers simultaneously using already existing antibody biologics.

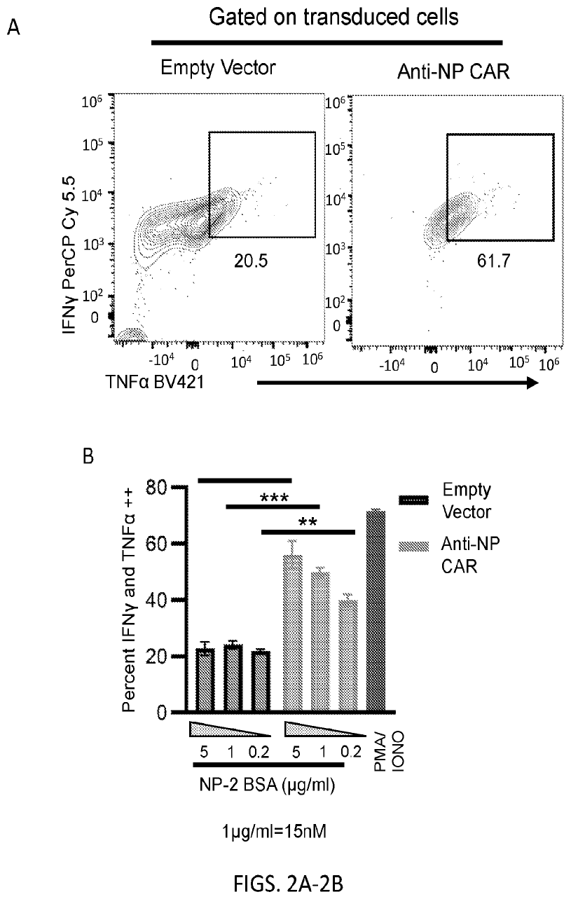

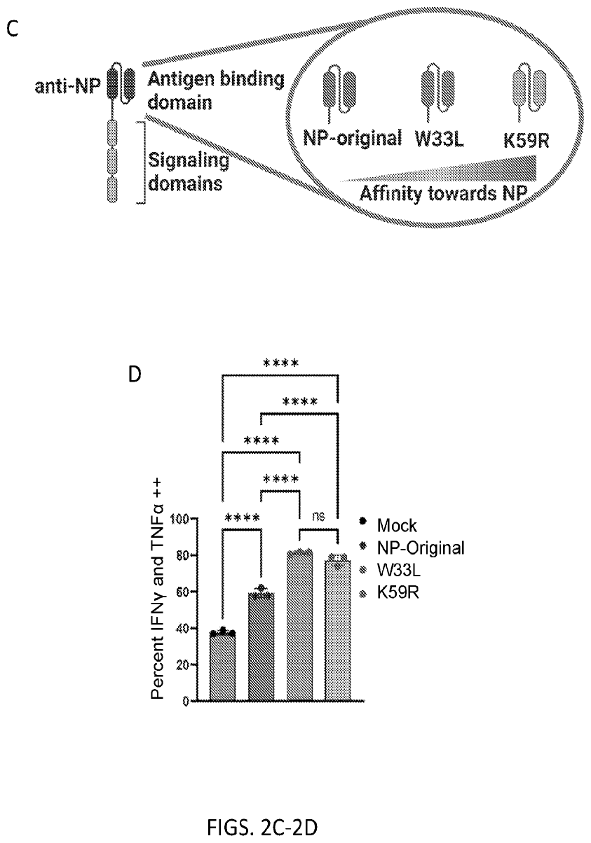

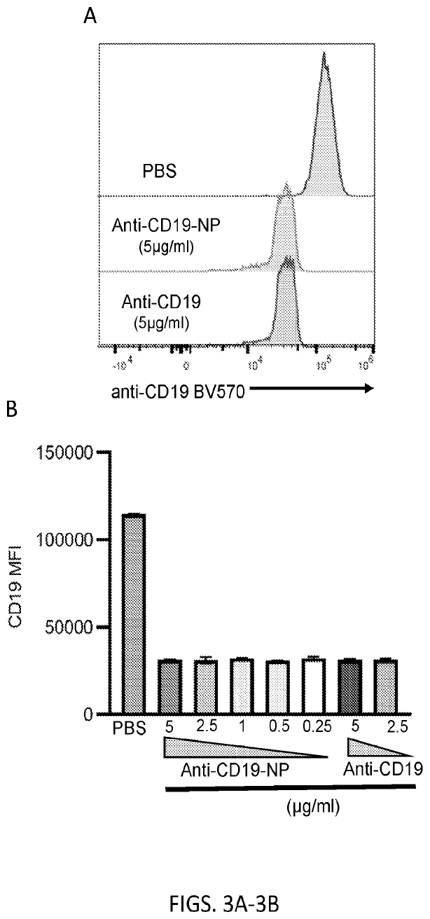

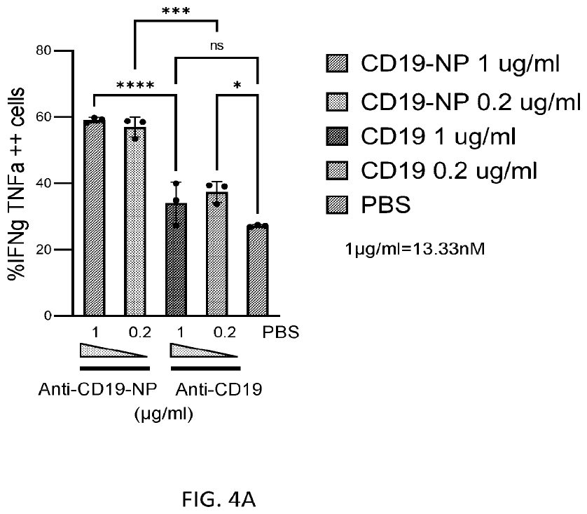

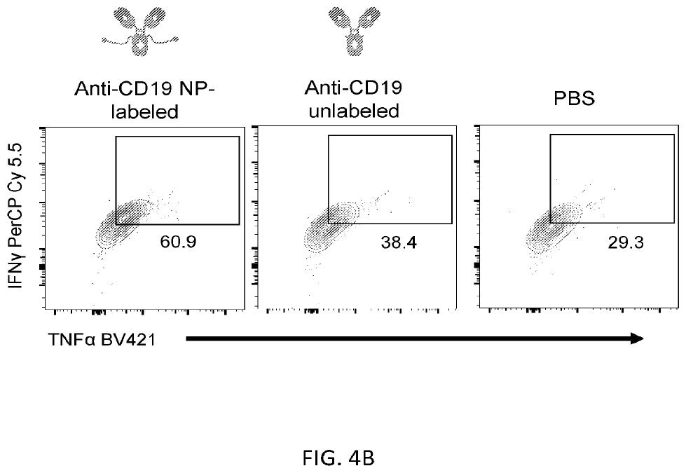

Modified nitrophenols (NP) are a known class of small molecule haptens that are immunogenic when conjugated to an antigen. We have further optimized the design of the antigen recognition domain of NP-specific CAR (anti-NP CAR) by introducing point mutations that allow high affinity NP-binding by the CAR, thereby lowering the need to extensively modify the antibodies while preserving their own biological activity. In validation studies, expression of anti-NP CAR in activated CD8 T cells enhanced IFNγ and TNFα production in an antigen dose-dependent manner, providing key evidence for the feasibility of this approach (FIG. 2). As shown in FIG. 3, NP modification doesn't alter antibody function. As shown in FIG. 4, the anti-NP CARs can be stimulated with NP labeled antibodies.

B16F10 melanoma cells expresses PDL-1 at high levels (data not shown). We stably transduced B16F10 cells to express human CD19 to compare the efficacy with current CAR-T therapy. Using these B16F10 cells as a model we tested the cytotoxicity of MASS-CAR in vitro. As shown in FIG. 5, MASS-CAR are switchable and are efficient in killing cancer cells in vitro.

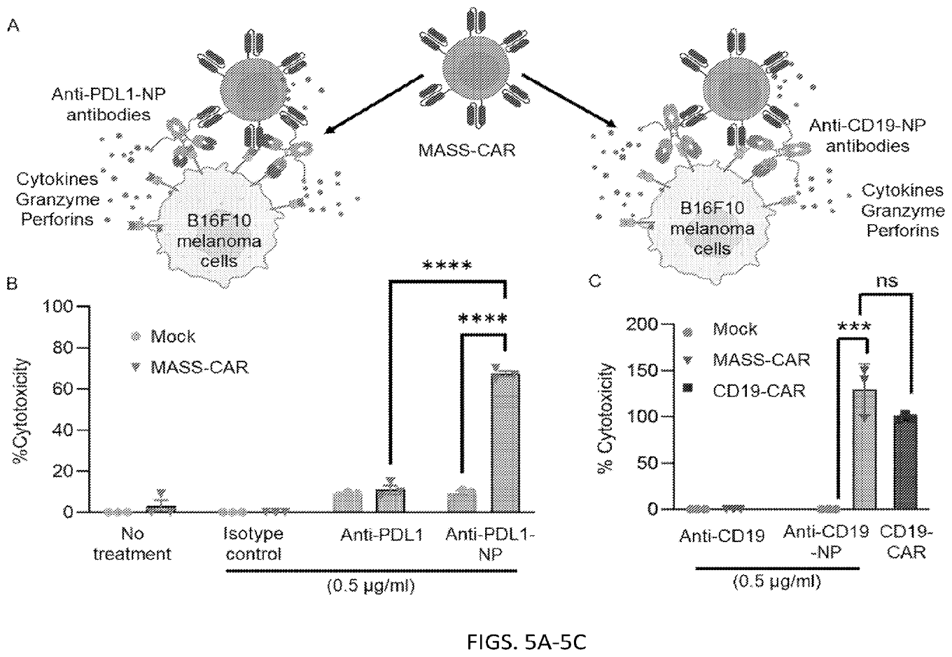

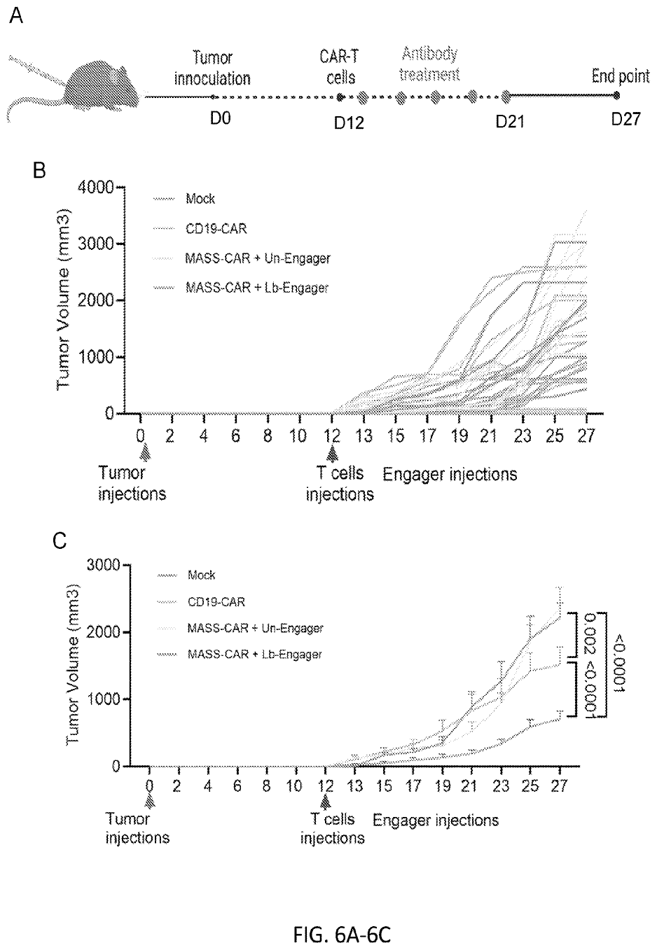

Next, the effect of MASS-CAR was examined, in vivo. MASS-CAR efficiently targets tumor cells in vivo. FIG. 6A. illustrates a timeline for in vivo MASS-CAR testing with CAR T cells and an engager (antibody conjugated with NP). As shown in FIGS. 6B and 6C, MASS-CAR was more effective at reducing tumor growth than CD-19 CAR T cells alone or with CD-19 CAR T cells and antibody not conjugated with NP.

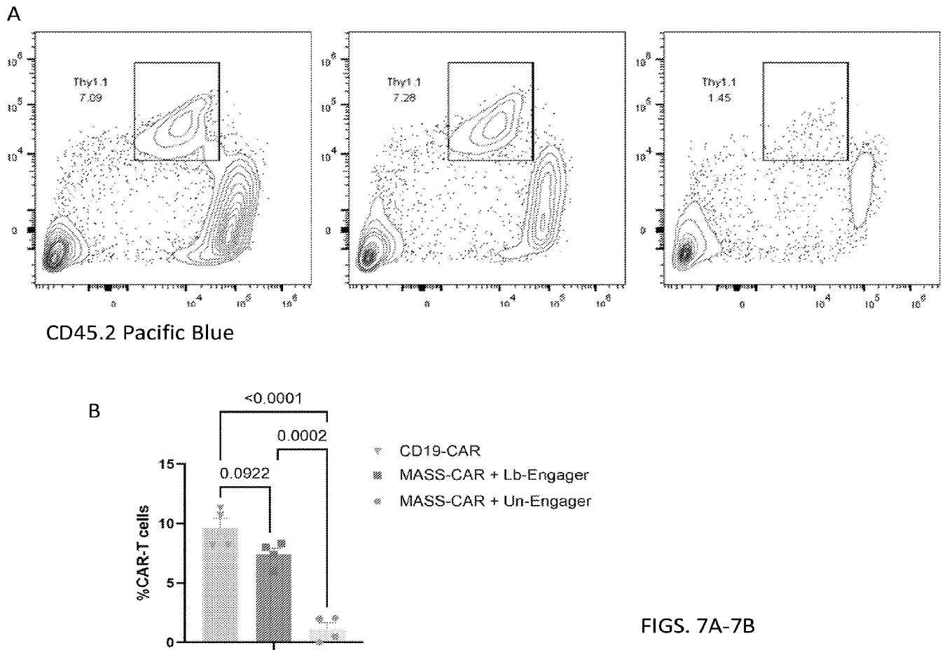

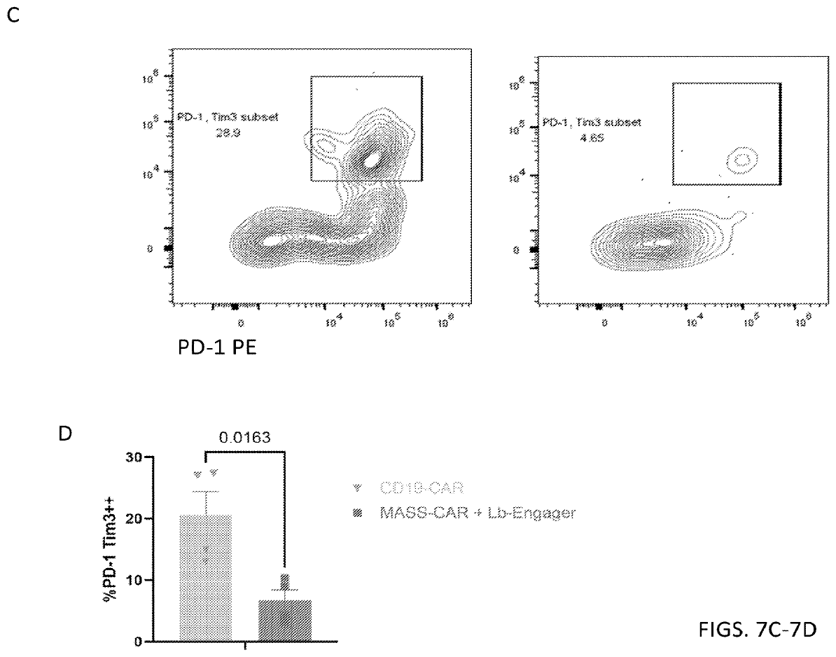

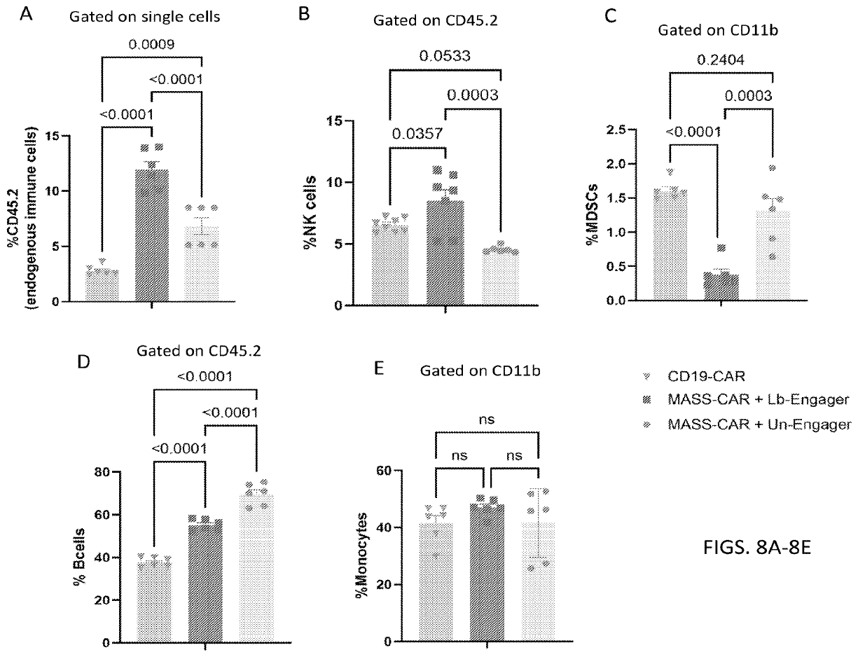

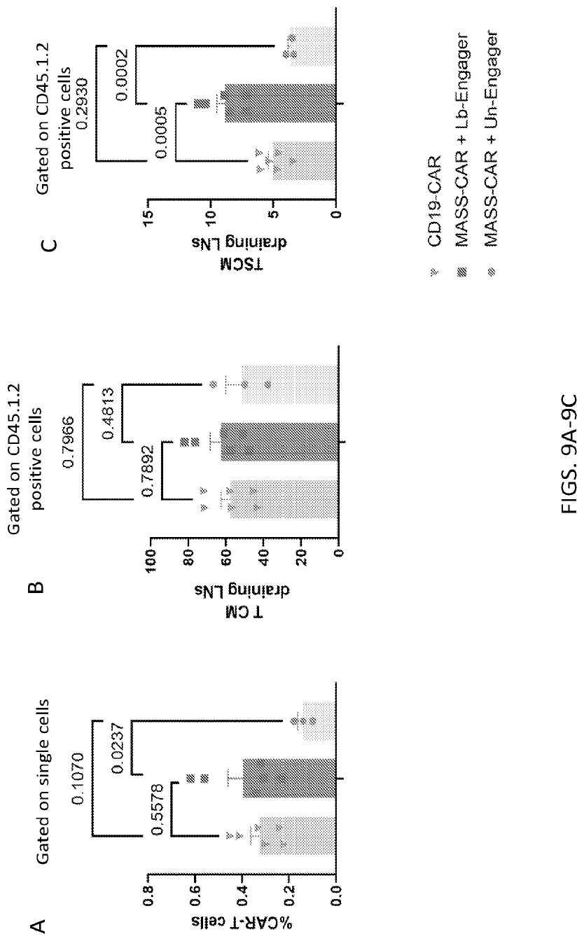

FIG. 7 illustrates recruitment of MASS-CARs at tumors in the presence NP-labelled engagers (Lb-Engager). FIGS. 8 and 9 illustrate that MASS-CARs with labelled engagers enhance the recruitment of endogenous immune cells to tumors.

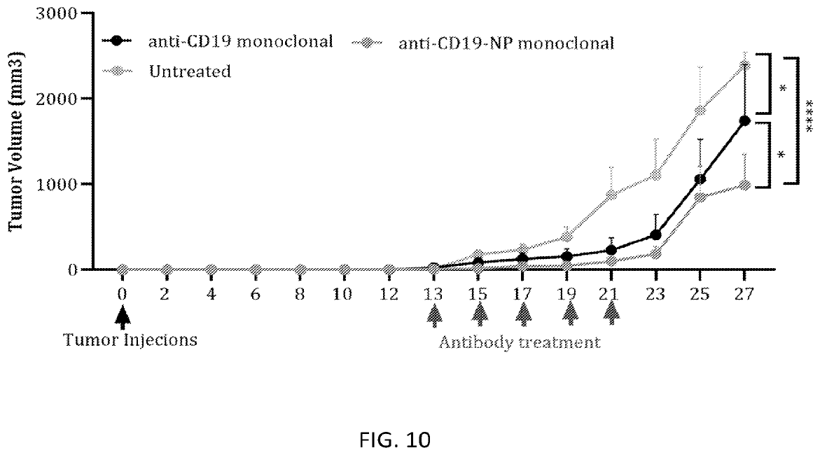

FIG. 10 illustrates that anti-CD19 antibody-hapten conjugate is more efficacious than anti-CD19 antibody in reducing tumor growth.

### REFERENCES

1 June, C. H., O'Connor, R. S., Kawalekar, O. U., Ghassemi, S. & Milone, M. C. CAR T cell immunotherapy for human cancer. Science 359, 1361-1365 (2018).
2 Sterner, R. C. & Sterner, R. M. CAR-T cell therapy: current limitations and potential strategies. Blood Cancer J 11, 69 (2021).
3 Ali, S. A. et al. T cells expressing an anti-B-cell maturation antigen chimeric antigen receptor cause remissions of multiple myeloma. Blood 128, 1688-1700 (2016).
4 Grupp, S. A. et al. Chimeric antigen receptor-modified T cells for acute lymphoid leukemia. N Engl J Med 368, 1509-1518 (2013).
Locke, F. L. et al. Long-term safety and activity of axicabtagene ciloleucel in refractory large B-cell lymphoma (ZUMA-1): a single-arm, multicentre, phase 1-2 trial. Lancet Oncol 20, 31-42 (2019).
6 Porter, D. L. et al. Chimeric antigen receptor T cells persist and induce sustained remissions in relapsed refractory chronic lymphocytic leukemia. Sci Transl Med 7, 303ra139 (2015).
7 Morgan, R. A. et al. Cancer regression in patients after transfer of genetically engineered lymphocytes. Science 314, 126-129 (2006).
8 Bloemberg, D. et al. A High-Throughput Method for Characterizing Novel Chimeric Antigen Receptors in Jurkat Cells. Mol Ther Methods Clin Dev 16, 238-254 (2020).
9 Chen, J. et al. NR4A transcription factors limit CAR T cell function in solid tumours. Nature 567, 530-534 (2019).
10 Horton, H. M. et al. Potent in vitro and in vivo activity of an Fc-engineered anti-CD 19 monoclonal antibody against lymphoma and leukemia. Cancer Res 68, 8049-8057 (2008).

INFORMAL SEQUENCE LISTING

| SEQ ID | | |
|---|---|---|
| NO: | Description | Sequence |
| 1 | NP CAR with | MLRLLLALNLFPSIQVTGGSSqavvtqesalttspgetvtltcrsstgavttsnyan |
| | CD3 Zeta and | wvqekpdhlftgliggtnnrapgvparfsgslignkaaltitgaqtedeaiyfcalwysnhwvf |
| | CD28 | gggtkltvleggggsggggsggggsqvqlqqpgaelvkpgasvklsckasgytftsywmhw |
| | Signaling | vkqrpgrglewigridpnsggtkynekfkskatltvdkpsstaymqlssltsedsavyycargl |
| | domains | ydgnwyfdvwgtgttvtvssesqsfpnvfplvscesplsdknlvaAAAGTTTTPAPR |
| | (human CAR) | PPTPAPTIASQPLSLRPEACRPAAGGAVHTRGLDFACDRRPPS |
| | | KPFWVLVVVGGVLACYSLLVTVAFIIFWVRSKRSRLLHSDYM |
| | | NMTPRRPGPTRKHYQPYAPPRDFAAYRSASLRVKFSRSADAP |
| | | AYQQGQNQLYNELNLGRREEYDVLDKRRGRDPEMGGKPQR |
| | | RKNPQEGLYNELQKDKMAEAYSEIGMKGERRRGKGHDGLY |
| | | QGLSTATKDTYDALHMQALPPRAS |
| 2 | Anti-NP | qavvtqesalttspgetvtltcrsstgavttsnyanwvqekpdhlftgliggtnnrapgvparfsg |
| | ScFV | slignkaaltitgaqtedeaiyfcalwysnhwvfgggtkltvleggggsggggsggggsqvqlq |
| | | qpgaelvkpgasvklsckasgytftsywmhwvkqrpgrglewigridpnsggtkynekfks |
| | | katltvdkpsstaymqlssltsedsavyycarglydgnwyfdvwgtgttvtvssesqsfpnvfp |
| | | lvscesplsdknlva |
| 3 | Anti-NP | qavvtqesalttspgetvtltcrsstgavttsnyanwvqekpdhlftgliggtnnrapgvparfsg |
| | ScFV | slignkaaltitgaqtedeaiyfcalwysnhwvfgggtkltvleggggsggggsggggsqvqlq |
| | W33L mutant | qpgaelvkpgasvklsckasgytftsylmhwvkqrpgrglewigridpnsggtkynekfksk |
| | | atltvdkpsstaymqlssltsedsavyycarglydgnwyfdvwgtgttvtvssesqsfpnvfpl |
| | | vscesplsdknlva |
| 4 | Anti-NP | qavvtqesalttspgetvtltcrsstgavttsnyanwvqekpdhlftgliggtnnrapgvparfsg |
| | ScFV K59R | slignkaaltitgaqtedeaiyfcalwysnhwvfgggtkltvleggggsggggsggggsqvqlq |
| | mutant | qpgaelvkpgasvklsckasgytftsywmhwvkqrpgrglewigridpnsggtrynekfks |
| | | katltvdkpsstaymqlssltsedsavyycarglydgnwyfdvwgtgttvtvssesqsfpnvfp |
| | | lvscesplsdknlva |
| 5 | NP CAR with | MLRLLLALNLFPSIQVTGGSSqavvtqesalttspgetvtltersstgavttsnyan |
| | CD3 Zeta and | wvqekpdhlftgliggtnnrapgvparfsgslignkaaltitgaqtedeaiyfcalwysnhwvf |
| | CD28 | gggtkltvleggggsggggsggggsqvqlqqpgaelvkpgasvklsckasgytftsylmhw |
| | Signaling | vkqrpgrglewigridpnsggtkynekfkskatltvdkpsstaymqlssltsedsavyycargl |
| | domains; | ydgnwyfdvwgtgttvtvssesqsfpnvfplvscesplsdknlvaAAAGTTTTPAPR |
| | W33L mutant | PPTPAPTIASQPLSLRPEACRPAAGGAVHTRGLDFACDRRPPS |
| | (human CAR) | KPFWVLVVVGGVLACYSLLVTVAFIIFWVRSKRSRLLHSDYM |
| | | NMTPRRPGPTRKHYQPYAPPRDFAAYRSASLRVKFSRSADAP |
| | | AYQQGQNQLYNELNLGRREEYDVLDKRRGRDPEMGGKPQR |
| | | RKNPQEGLYNELQKDKMAEAYSEIGMKGERRRGKGHDGLY |
| | | QGLSTATKDTYDALHMQALPPRAS |
| 6 | NP CAR with | MLRLLLALNLFPSIQVTGGSSqavvtqesalttspgetvtltersstgavttsnyan |
| | CD3 Zeta and | wvqekpdhlftgliggtnnrapgvparfsgslignkaaltitgaqtedeaiyfcalwysnhwvf |
| | CD28 | gggtkltvleggggsggggsggggsqvqlqqpgaelvkpgasvklsckasgytftsywmhw |
| | Signaling | vkqrpgrglewigridpnsggtrynekfkskatltvdkpsstaymqlssltsedsavyycargl |
| | domains; | ydgnwyfdvwgtgttvtvssesqsfpnvfplvscesplsdknlvaAAAGTTTTPAPR |
| | K59R mutant | PPTPAPTIASQPLSLRPEACRPAAGGAVHTRGLDFACDRRPPS |
| | (human CAR) | KPFWVLVVVGGVLACYSLLVTVAFIIFWVRSKRSRLLHSDYM |
| | | NMTPRRPGPTRKHYQPYAPPRDFAAYRSASLRVKFSRSADAP |
| | | AYQQGQNQLYNELNLGRREEYDVLDKRRGRDPEMGGKPQR |
| | | RKNPQEGLYNELQKDKMAEAYSEIGMKGERRRGKGHDGLY |
| | | QGLSTATKDTYDALHMQALPPRAS |
| 7 | NP CAR with | MLRLLLALNLFPSIQVTGGSSqavvtqesalttspgetvtltersstgavttsnyan |
| | CD3 Zeta and | wvqekpdhlftgliggtnnrapgvparfsgslignkaaltitgaqtedeaiyfcalwysnhwvf |
| | 4-1BB | gggtkltvleggggsggggsggggsqvqlqqpgaelvkpgasvklsckasgytftsywmhw |
| | Signaling | vkqrpgrglewigridpnsggtkynekfkskatltvdkpsstaymqlssltsedsavyycargl |
| | domains | ydgnwyfdvwgtgttvtvssesqsfpnvfplvscesplsdknlvaAAAGTTTTPAPR |
| | (human CAR) | PPTPAPTIASQPLSLRPEACRPAAGGAVHTRGLDFACDRRPPS |
| | | KPFWVLVVVGGVLACYSLLVTVAFIIFWVRKRGRKKLLYIFK |
| | | QPFMRPVQTTQEEDGCSCRFPEEEEGGCELASLRVKFSRSADA |
| | | PAYQQGQNQLYNELNLGRREEYDVLDKRRGRDPEMGGKPQ |
| | | RRKNPQEGLYNELQKDKMAEAYSEIGMKGERRRGKGHDGL |
| | | YQGLSTATKDTYDALHMQALPPRAS |
| 8 | NP CAR with | MLRLLLALNLFPSIQVTGGSSqavvtqesalttspgetvtltersstgavttsnyan |
| | CD3 Zeta and | wvqekpdhlftgliggtnnrapgvparfsgslignkaaltitgaqtedeaiyfcalwysnhwvf |
| | 4-1BB | gggtkltvleggggsggggsggggsqvqlqqpgaelvkpgasvklsckasgytftsylmhw |
| | Signaling | vkqrpgrglewigridpnsggtkynekfkskatltvdkpsstaymqlssltsedsavyycargl |
| | domains; | ydgnwyfdvwgtgttvtvssesqsfpnvfplvscesplsdknlvaAAAGTTTTPAPR |
| | W33L mutant | PPTPAPTIASQPLSLRPEACRPAAGGAVHTRGLDFACDRRPPS |
| | (human CAR) | KPFWVLVVVGGVLACYSLLVTVAFIIFWVRKRGRKKLLYIFK |
| | | QPFMRPVQTTQEEDGCSCRFPEEEEGGCELASLRVKFSRSADA |
| | | PAYQQGQNQLYNELNLGRREEYDVLDKRRGRDPEMGGKPQ |
| | | RRKNPQEGLYNELQKDKMAEAYSEIGMKGERRRGKGHDGL |
| | | YQGLSTATKDTYDALHMQALPPRAS |
| 9 | NP CAR with | MLRLLLALNLFPSIQVTGGSSqavvtqesalttspgetvtltersstgavttsnyan |
| | CD3 Zeta and | wvqekpdhlftgliggtnnrapgvparfsgslignkaaltitgaqtedeaiyfcalwysnhwvf |
| | 4-1BB | gggtkltvleggggsggggsggggsqvqlqqpgaelvkpgasvklsckasgytftsywmhw |
| | Signaling | vkqrpgrglewigridpnsggtrynekfkskatltvdkpsstaymqlssltsedsavyycargl |
| | domains; | ydgnwyfdvwgtgttvtvssesqsfpnvfplvscesplsdknlvaAAAGTTTTPAPR |
| | K59R mutant | PPTPAPTIASQPLSLRPEACRPAAGGAVHTRGLDFACDRRPPS |
| | (human CAR) | KPFWVLVVVGGVLACYSLLVTVAFIIFWVRKRGRKKLLYIFK |
| | | QPFMRPVQTTQEEDGCSCRFPEEEEGGCELASLRVKFSRSADA |
| | | PAYQQGQNQLYNELNLGRREEYDVLDKRRGRDPEMGGKPQ |
| | | RRKNPQEGLYNELQKDKMAEAYSEIGMKGERRRGKGHDGL |
| | | YQGLSTATKDTYDALHMQALPPRAS |
| 10 | NP CAR with | MLRLLLALNLFPSIQVTGGSSqavvtqesalttspgetvtltersstgavttsnyan |
| | CD3 Zeta, 4- | wvqekpdhlftgliggtnnrapgvparfsgslignkaaltitgaqtedeaiyfcalwysnhwvf |
| | 1BB and | gggtkltvleggggsggggsggggsqvqlqqpgaelvkpgasvklsckasgytftsywmhw |
| | CD28 | vkqrpgrglewigridpnsggtrynekfkskatltvdkpsstaymqlssltsedsavyycargl |
| | Signaling | ydgnwyfdvwgtgttvtvssesqsfpnvfplvscesplsdknlvaAAAGTTTTPAPR |
| | domains | PPTPAPTIASQPLSLRPEACRPAAGGAVHTRGLDFACDRRPKR |
| | (human CAR) | GRKKLLYIFKQPFMRPVQTTQEEDGCSCRFPEEEEGGCELGSG |
| | | SPSKPFWVLVVVGGVLACYSLLVTVAFIIFWVRSKRSRLLHSD |
| | | YMNMTPRRPGPTRKHYQPYAPPRDFAAYRSASLRVKFSRSAD |
| | | APAYQQGQNQLYNELNLGRREEYDVLDKRRGRDPEMGGKP |
| | | QRRKNPQEGLYNELQKDKMAEAYSEIGMKGERRRGKGHDG |
| | | LYQGLSTATKDTYDALHMQALPPRAS |
| 11 | NP CAR with | MLRLLLALNLFPSIQVTGGSSqavvtqesalttspgetvtltcrsstgavttsnyan |
| | CD3 Zeta, 4- | wvqekpdhlftgliggtnnrapgvparfsgslignkaaltitgaqtedeaiyfcalwysnhwvf |
| | 1BB and | gggtkltvleggggsggggsggggsqvqlqqpgaelvkpgasvklsckasgytftsylmhw |
| | CD28 | vkqrpgrglewigridpnsggtkynekfkskatltvdkpsstaymqlssltsedsavyycargl |
| | Signaling | ydgnwyfdvwgtgttvtvssesqsfpnvfplvscesplsdknlvaAAAGTTTTPAPR |
| | domains; | PPTPAPTIASQPLSLRPEACRPAAGGAVHTRGLDFACDRRPKR |
| | W33L mutant | GRKKLLYIFKQPFMRPVQTTQEEDGCSCRFPEEEEGGCELGSG |
| | (human CAR) | SPSKPFWVLVVVGGVLACYSLLVTVAFIIFWVRSKRSRLLHSD |
| | | YMNMTPRRPGPTRKHYQPYAPPRDFAAYRSASLRVKFSRSAD |
| | | APAYQQGQNQLYNELNLGRREEYDVLDKRRGRDPEMGGKP |
| | | QRRKNPQEGLYNELQKDKMAEAYSEIGMKGERRRGKGHDG |
| | | LYQGLSTATKDTYDALHMQALPPRAS |
| 12 | NP CAR with | MLRLLLALNLFPSIQVTGGSSqavvtqesalttspgetvtltersstgavttsnyan |
| | CD3 Zeta, 4- | wvqekpdhlftgliggtnnrapgvparfsgslignkaaltitgaqtedeaiyfcalwysnhwvf |
| | 1BB and | ggtkltvleggggsggggsggggsqvqlqqpgaelvkpgasvklsckasgytftsywmhw |
| | CD28 | vkqrpgrglewigridpnsggtrynekfkskatltvdkpsstaymqlssltsedsavyycargl |
| | Signaling | ydgnwyfdvwgtgttvtvssesqsfpnvfplvscesplsdknlvaAAAGTTTTPAPR |
| | domains; | PPTPAPTIASQPLSLRPEACRPAAGGAVHTRGLDFACDRRPKR |
| | K59R mutant | GRKKLLYIFKQPFMRPVQTTQEEDGCSCRFPEEEEGGCELGSG |
| | (human CAR) | SPSKPFWVLVVVGGVLACYSLLVTVAFIIFWVRSKRSRLLHSD |
| | | YMNMTPRRPGPTRKHYQPYAPPRDFAAYRSASLRVKFSRSAD |
| | | APAYQQGQNQLYNELNLGRREEYDVLDKRRGRDPEMGGKP |
| | | QRRKNPQEGLYNELQKDKMAEAYSEIGMKGERRRGKGHDG |
| | | LYQGLSTATKDTYDALHMQALPPRAS |
| 13 | NP CAR: | Malpvtalllplalllhaarpgkpipnpllgldstqavvtqesalttspgetvtltcrsstgavttsn |
| | with CD3 zeta | yanwvqekpdhlftgliggtnnrapgvparfsgslignkaaltitgaqtedeaiyfcalwysnhw |
| | and CD28 | vfgggtkltvleggggsggggsggggsqvqlqqpgaelvkpgasvklsckasgytftsywm |
| | signaling | hwvkqrpgrglewigridpnsggtkynekfkskatltvdkpsstaymqlssltsedsavyyca |
| | domains | rglydgnwyfdvwgtgttvtvssesqsfpnvfplvscesplsdknlvaIEFMYPPPYLD |
| | (mouse CAR) | NERSNGTIIHIKEKHLCHTQSSPKLFWALVVVAGVLFCYGLLV |
| | | TVALCVIWTNSRRNRGGQSDYMNMTPRRPGLTRKPYQPYAP |
| | | ARDFAAYRPRAKFSRSAETAANLQDPNQLYNELNLGRREEY |
| | | DVLEKKRARDPEMGGKQQRRRNPQEGVYNALQKDKMAEAY |
| | | SEIGTKGERRRGKGHDGLYQGLSTATKDTYDALHMQTLAPR |
| | | GSG |
| 14 | NP CAR: | Malpvtalllplalllhaarpgkpipnpllgldstqavvtqesalttspgetvtltcrsstgavttsn |
| | with CD3 zeta | yanwvqekpdhlftgliggtnnrapgvparfsgslignkaaltitgaqtedeaiyfcalwysnhw |
| | and CD28 | vfgggtkltvleggggsggggsggggsqvqlqqpgaelvkpgasvklsckasgytftsylmh |
| | signaling | wvkqrpgrglewigridpnsggtkynekfkskatltvdkpsstaymqlssltsedsavyycar |
| | domains; | glydgnwyfdvwgtgttvtvssesqsfpnvfplvscesplsdknlvaIEFMYPPPYLD |
| | W33L mutant | NERSNGTIIHIKEKHLCHTQSSPKLFWALVVVAGVLFCYGLLV |
| | (mouse CAR) | TVALCVIWTNSRRNRGGQSDYMNMTPRRPGLTRKPYQPYAP |
| | | ARDFAAYRPRAKFSRSAETAANLQDPNQLYNELNLGRREEY |
| | | DVLEKKRARDPEMGGKQQRRRNPQEGVYNALQKDKMAEAY |
| | | SEIGTKGERRRGKGHDGLYQGLSTATKDTYDALHMQTLAPR |
| | | GSG |
| 15 | NP CAR: | Malpvtalllplalllhaarpgkpipnpllgldstqavvtqesalttspgetvtltcrsstgavttsn |
| | with CD3 zeta | yanwvqekpdhlftgliggtnnrapgvparfsgslignkaaltitgaqtedeaiyfcalwysnhw |
| | and CD28 | vfgggtkltvleggggsggggsggggsqvqlqqpgaelvkpgasvklsckasgytftsywm |
| | signaling | hwvkqrpgrglewigridpnsggtrynekfkskatltvdkpsstaymqlssltsedsavyyca |
| | domains; | rglydgnwyfdvwgtgttvtvssesqsfpnvfplvscesplsdknlvaIEFMYPPPYLD |
| | K59R mutant | NERSNGTIIHIKEKHLCHTQSSPKLFWALVVVAGVLFCYGLLV |
| | (mouse CAR) | TVALCVIWTNSRRNRGGQSDYMNMTPRRPGLTRKPYQPYAP |
| | | ARDFAAYRPRAKFSRSAETAANLQDPNQLYNELNLGRREEY |
| | | DVLEKKRARDPEMGGKQQRRRNPQEGVYNALQKDKMAEAY |
| | | SEIGTKGERRRGKGHDGLYQGLSTATKDTYDALHMQTLAPR |
| | | GSG |
| 16 | CD3 zeta | LRVKFSRSADAPAYQQGQNQLYNELNLGRREEYDVLDKRRG |
| | signaling | RDPEMGGKPQRRKNPQEGLYNELQKDKMAEAYSEIGMKGER |
| | domain | RRGKGHDGLYQGLSTATKDTYDALHMQALPPR |
| | (human) | |
| 17 | CD28 | GSGSPSKPFWVLVVVGGVLACYSLLVTVAFIIFWVRSKRSRLL |
| | signaling | HSDYMNMTPRRPGPTRKHYQPYAPPRDFAAYRS |
| | domain | |
| | (human) | |
| 18 | 4-1BB | KRGRKKLLYIFKQPFMRPVQTTQEEDGCSCRFPEEEEGGCEL |
| | signaling | |
| | domain | |
| | (human) | |
| 19 | CD3 zeta | RAKFSRSAETAANLQDPNQLYNELNLGRREEYDVLEKKRAR |
| | signaling | DPEMGGKQQRRRNPQEGVYNALQKDKMAEAYSEIGTKGER |
| | domain | RRGKGHDGLYQGLSTATKDTYDALHMQTLAPRGSG |
| | (mouse) | |
| 20 | CD28 | FWALVVVAGVLFCYGLLVTVALCVIWTNSRRNRGGQSDYM |
| | signaling | NMTPRRPGLTRKPYQPYAPARDFAAYRP |
| | domain | |
| | (mouse) | |
| 21 | 4-1BB | KWIRKKFPHIFKQPFKKTTGAAQEEDACSCRCPQEEEGGGGG |
| | signaling | YE |
| | domain | |
| | (mouse) | |
| 22 | NP CAR: | MLRLLLALNLFPSIQVTGGSSGKPIPNPLLGLDSTQAVVTQES |
| | with CD3 zeta | ALTTSPGETVTLTCRSSTGAVTTSNYANWVQEKPDHLFTGLIG |
| | (italicized), | GTNNRAPGVPARFSGSLIGNKAALTITGAQTEDEAIYFCALWY |
| | 41BB | SNHWVFGGGTKLTVLEGGGGSGGGGSGGGGSQVQLQQPGAE |
| | (bolded) and | LVKPGASVKLSCKASGYTFTSYWMHWVKQRPGRGLEWIGRIDPN |
| | CD28 | SGGTRYNEKFKSKATLTVDKPSSTAYMQLSSLTSEDSAVYYCARGL |
| | (bolded and | YDGNWYFDVWGTGTTVTVSSESQSFPNVFPLVSCESPLSDKNLVA |
| | underlined) | ESKYGPPCPSCPPSKPFWVLVVVGGVLACYSLLVTVAFIIF |
| | signaling | WVRSKRSRLLHSDYMNMTPRRPGPTRKHYQPYAPPRDFA |
| | domains with | AYRSKRGRKKLLYIFKQPFMRPVQTTQEEDGCSCRFPEEE |
| | Ig hinge | EGGCELGSLRVKFSRSADAPAYQQGQNQLYNELNLGRREEYDV |
| | region | LDKRRGRDPEMGGKPQRRKNPQEGLYNELQKDKMAEAYSEIGM |
| | (underlined); | KGERRRGKGHDGLYQGLSTATKDTYDALHMQALPPR |
| | K59R mutant | |
| | (italicized | |
| | and | |
| | underlined) | |
| | (Human | |
| | CAR) | |
| 23 | NP CAR: | MLRLLLALNLFPSIQVTGGSSGKPIPNPLLGLDSTQAVVTQES |
| | with CD3 zeta | ALTTSPGETVTLTCRSSTGAVTTSNYANWVQEKPDHLFTGLIG |
| | (italicized), | GTNNRAPGVPARFSGSLIGNKAALTITGAQTEDEAIYFCALWY |
| | 41BB | SNHWVFGGGTKLTVLEGGGGSGGGGSGGGGSQVQLQQPGAE |
| | (bolded) and | LVKPGASVKLSCKASGYTFTSYWMHWVKQRPGRGLEWIGRIDPN |
| | CD28 | SGGTRYNEKFKSKATLTVDKPSSTAYMQLSSLTSEDSAVYYCARGL |
| | (bolded and | YDGNWYFDVWGTGTTVTVSSESQSFPNVEPLVSCESPLSDKNLVA |
| | underlined) | ESKYGPPCPPCPFWALVVVAGVLFCYGLLVTVALCVIWINS |
| | signaling | RRNRGGQSDYMNMTPRRPGLTRKPYQPYAPARDFAAYRP |
| | domains with | KWIRKKFPHIFKQPFKKTTGAAQEEDACSCRCPQEEEGG |
| | Ig hinge | GGGYELRAKFSRSAETAANLQDPNQLYNELNLGRREEYDVLEKK |
| | region | RARDPEMGGKQQRRRNPQEGVYNALQKDKMAEAYSEIGTKGER |
| | (underlined); | RRGKGHDGLYQGLSTATKDTYDALHMQTLAPR |
| | K59R mutant | |
| | (italicized | |
| | and | |
| | underlined) | |
| | (Mouse CAR) | |
| 24 | CD28 leader | MLRLLLALNLFPSIQVTGGSS |
| | sequence | |
| | (Human) | |
| 25 | CD28 leader | MALPVTALLLPLALLLHAARP |
| | sequence | |
| | (Mouse) | |
| 26 | IgG4 hinge | ESKYGPPCPSCP |
| | region | |
| | (human) | |
| 27 | IgG4 hinge | ESKYGPPCPPCP |
| | region | |
| | (Mouse) | |
| 28 | CD8 hinge | AAAGTTTTPAPRPPTPAPTIASQPLSLRPEACRPAAGGAVHTR |
| | region | GLDFACDRRP |
| | (human) | |
| 29 | CD8 hinge | IEFMYPPPYLDNERSNGTIIHIKEKHLCHTQSSPKL |
| | region | |
| | (mouse) | |
| 30 | V5 tag | gkpipnpllgldst |

1. An engineered polynucleotide encoding a chimeric antigen receptor (CAR) comprising an extracellular antigen binding region that binds to a hapten; and an intracellular immune cell activation domain.

2. The engineered polynucleotide of claim 1, wherein the hapten is a nitrophenol (NP).

3. The engineered polynucleotide of claim 2, wherein the NP is 4-hydroxy-3-nitrophenylacetyl.

4. The engineered polynucleotide of claim 3, wherein the antigen binding region comprises a sequence having at least 95% identity to SEQ ID NO: 2, SEQ ID NO: 3, or SEQ ID NO: 4.

5. The engineered polynucleotide of claim 1, wherein the immune cell activation domain comprises at least two of a CD28 signaling domain, a 4-1BB domain, and a CD3ζ signaling domain.

6. The engineered polynucleotide of claim 1, wherein the CAR comprises a sequence having at least 95% identity to SEQ ID NO: 1, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 22.

7. A construct comprising the engineered polynucleotide of claim 1 operably linked to a heterologous promoter.

8. A CAR encoded by the engineered polynucleotide of claim 1.

9. A host cell comprising the engineered polynucleotide of claim 1.

10. The host cell of claim 9, wherein the host cell is an immune cell.

11. (canceled)

12. A modified antibody molecule, wherein the antibody molecule is conjugated to at least one hapten group.

13. The modified antibody molecule of claim 12, wherein the hapten is a nitrophenol.

14. The modified antibody molecule of claim 13, wherein the nitrophenol is 4-hydroxy-3-nitrophenylacetyl.

15. The modified antibody molecule claim 12, wherein the antibody molecule comprises a binding region specific to a tumor specific antigen or an antigen associated with an autoimmune disease.

16. The modified antibody molecule of claim 15, wherein the tumor specific antigen is CD19 or PD1.

17. The modified antibody molecule of claim 12, wherein the antibody molecule is selected from a monoclonal antibody, a single chain antibody, a nanobody, a T cell engager, and a NK cell engager.

18. A kit comprising:
the engineered polynucleotide of any one of claim 1; and
a modified antibody molecule, wherein the antibody molecule is conjugated to at least one hapten group;
wherein the CAR antigen binding region binds to the hapten group conjugated to the modified antibody molecule.

19. A method of treating a cancer or an autoimmune disease in a subject, the method comprising administering to the subject:
a therapeutically effective amount of a first pharmaceutical composition comprising the modified antibody molecule of claim 12; and a pharmaceutically acceptable carrier.

20. The method of claim 19, further comprising administering to the subject:
a therapeutically effective amount of a second pharmaceutical composition comprising the host cell of claim 9; and a pharmaceutically acceptable carrier;
wherein the CAR antigen binding region binds to the hapten group conjugated to the modified antibody molecule.

21. The method of claim 20, wherein the second pharmaceutical composition is administered before the first pharmaceutic composition.


SEQUENCE LISTING (30 sequences)

SEQ ID NO: 1 - protein, 534 aa, synthetic construct
MLRLLLALNLFPSIQVTGGSSQAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYWMHWVKQRPGRGLEWIGRIDPNSGGTKYNEKFKSKATLTVDKPSSTAYMQLSSLTSEDSAVYYCARGLYDGNWYFDVWGTGTTVTVSSESQSFPNVFPLVSCESPLSDKNLVAAAAGTTTTPAPRPPTPAPTIASQPLSLRPEACRPAAGGAVHTRGLDFACDRRPPSKPFWVLVVVGGVLACYSLLVTVAFIIFWVRSKRSRLLHSDYMNMTPRRPGPTRKHYQPYAPPRDFAAYRSASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 2 - protein, 270 aa, synthetic construct
QAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYWMHWVKQRPGRGLEWIGRIDPNSGGTKYNEKFKSKATLTVDKPSSTAYMQLSSLTSEDSAVYYCARGLYDGNWYFDVWGTGTTVTVSSESQSFPNVFPLVSCESPLSDKNLVA

SEQ ID NO: 3 - protein, 270 aa, synthetic construct
QAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYLMHWVKQRPGRGLEWIGRIDPNSGGTKYNEKFKSKATLTVDKPSSTAYMQLSSLTSEDSAVYYCARGLYDGNWYFDVWGTGTTVTVSSESQSFPNVFPLVSCESPLSDKNLVA

SEQ ID NO: 4 - protein, 270 aa, synthetic construct
QAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYWMHWVKQRPGRGLEWIGRIDPNSGGTRYNEKFKSKATLTVDKPSSTAYMQLSSLTSEDSAVYYCARGLYDGNWYFDVWGTGTTVTVSSESQSFPNVFPLVSCESPLSDKNLVA

SEQ ID NO: 5 - protein, 534 aa, synthetic construct
MLRLLLALNLFPSIQVTGGSSQAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYLMHWVKQRPGRGLEWIGRIDPNSGGTKYNEKFKSKATLTVDKPSSTAYMQLSSLTSEDSAVYYCARGLYDGNWYFDVWGTGTTVTVSSESQSFPNVFPLVSCESPLSDKNLVAAAAGTTTTPAPRPPTPAPTIASQPLSLRPEACRPAAGGAVHTRGLDFACDRRPPSKPFWVLVVVGGVLACYSLLVTVAFIIFWVRSKRSRLLHSDYMNMTPRRPGPTRKHYQPYAPPRDFAAYRSASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 6 - protein, 534 aa, synthetic construct
MLRLLLALNLFPSIQVTGGSSQAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYWMHWVKQRPGRGLEWIGRIDPNSGGTRYNEKFKSKATLTVDKPSSTAYMQLSSLTSEDSAVYYCARGLYDGNWYFDVWGTGTTVTVSSESQSFPNVFPLVSCESPLSDKNLVAAAAGTTTTPAPRPPTPAPTIASQPLSLRPEACRPAAGGAVHTRGLDFACDRRPPSKPFWVLVVVGGVLACYSLLVTVAFIIFWVRSKRSRLLHSDYMNMTPRRPGPTRKHYQPYAPPRDFAAYRSASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 7 - protein, 536 aa, synthetic construct
MLRLLLALNLFPSIQVTGGSSQAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYWMHWVKQRPGRGLEWIGRIDPNSGGTKYNEKFKSKATLTVDKPSSTAYMQLSSLTSEDSAVYYCARGLYDGNWYFDVWGTGTTVTVSSESQSFPNVFPLVSCESPLSDKNLVAAAAGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 8 - protein, 536 aa, synthetic construct
MLRLLLALNLFPSIQVTGGSSQAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYLMHWVKQRPGRGLEWIGRIDPNSGGTKYNEKFKSKATLTVDKPSSTAYMQLSSLTSEDSAVYYCARGLYDGNWYFDVWGTGTTVTVSSESQSFPNVFPLVSCESPLSDKNLVAAAAGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 9 - protein, 536 aa, synthetic construct
MLRLLLALNLFPSIQVTGGSSQAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYWMHWVKQRPGRGLEWIGRIDPNSGGTRYNEKFKSKATLTVDKPSSTAYMQLSSLTSEDSAVYYCARGLYDGNWYFDVWGTGTTVTVSSESQSFPNVFPLVSCESPLSDKNLVAAAAGTTTTPAPRPPTPAPTIASQPLSLRPEACRPAAGGAVHTRGLDFACDRRPPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 10 - protein, 580 aa, synthetic construct
MLRLLLALNLFPSIQVTGGSSQAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYWMHWVKQRPGRGLEWIGRIDPNSGGTRYNEKFKSKATLTVDKPSSTAYMQLSSLTSEDSAVYYCARGLYDGNWYFDVWGTGTTVTVSSESQSFPNVFPLVSCESPLSDKNLVAAAAGTTTTPAPRPPTPAPTIASQPLSLRPEACRPAAGGAVHTRGLDFACDRRPKRGRKKLLYIFKQPFMRPVQTTQEEDGCSCRFPEEEEGGCELGSGSPSKPFWVLVVVGGVLACYSLLVTVAFIIFWVRSKRSRLLHSDYMNMTPRRPGPTRKHYQPYAPPRDFAAYRSASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 11 - protein, 580 aa, synthetic construct
MLRLLLALNLFPSIQVTGGSSQAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYLMHWVKQRPGRGLEWIGRIDPNSGGTKYNEKFKSKATLTVDKPSSTAYMQLSSLTSEDSAVYYCARGLYDGNWYFDVWGTGTTVTVSSESQSFPNVFPLVSCESPLSDKNLVAAAAGTTTTPAPRPPTPAPTIASQPLSLRPEACRPAAGGAVHTRGLDFACDRRPKRGRKKLLYIFKQPFMRPVQTTQEEDGCSCRFPEEEEGGCELGSGSPSKPFWVLVVVGGVLACYSLLVTVAFIIFWVRSKRSRLLHSDYMNMTPRRPGPTRKHYQPYAPPRDFAAYRSASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 12 - protein, 580 aa, synthetic construct; identical to SEQ ID NO: 10

SEQ ID NO: 13 - protein, 525 aa, synthetic construct
MALPVTALLLPLALLLHAARPGKPIPNPLLGLDSTQAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYWMHWVKQRPGRGLEWIGRIDPNSGGTKYNEKFKSKATLTVDKPSSTAYMQLSSLTSEDSAVYYCARGLYDGNWYFDVWGTGTTVTVSSESQSFPNVFPLVSCESPLSDKNLVAIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSG

SEQ ID NO: 14 - protein, 525 aa, synthetic construct
MALPVTALLLPLALLLHAARPGKPIPNPLLGLDSTQAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYLMHWVKQRPGRGLEWIGRIDPNSGGTKYNEKFKSKATLTVDKPSSTAYMQLSSLTSEDSAVYYCARGLYDGNWYFDVWGTGTTVTVSSESQSFPNVFPLVSCESPLSDKNLVAIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSG

SEQ ID NO: 15 - protein, 525 aa, synthetic construct
MALPVTALLLPLALLLHAARPGKPIPNPLLGLDSTQAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYWMHWVKQRPGRGLEWIGRIDPNSGGTRYNEKFKSKATLTVDKPSSTAYMQLSSLTSEDSAVYYCARGLYDGNWYFDVWGTGTTVTVSSESQSFPNVFPLVSCESPLSDKNLVAIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSG

SEQ ID NO: 16 - protein, 114 aa, synthetic construct
LRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - protein, 76 aa, synthetic construct
GSGSPSKPFWVLVVVGGVLACYSLLVTVAFIIFWVRSKRSRLLHSDYMNMTPRRPGPTRKHYQPYAPPRDFAAYRS

SEQ ID NO: 18 - protein, 42 aa, synthetic construct
KRGRKKLLYIFKQPFMRPVQTTQEEDGCSCRFPEEEEGGCEL

SEQ ID NO: 19 - protein, 116 aa, synthetic construct
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSG

SEQ ID NO: 20 - protein, 68 aa, synthetic construct
FWALVVVAGVLFCYGLLVTVALCVIWTNSRRNRGGQSDYMNMTPRRPGLTRKPYQPYAPARDFAAYRP

SEQ ID NO: 21 - protein, 44 aa, synthetic construct
KWIRKKFPHIFKQPFKKTTGAAQEEDACSCRCPQEEEGGGGGYE

SEQ ID NO: 22 - protein, 547 aa, synthetic construct
MLRLLLALNLFPSIQVTGGSSGKPIPNPLLGLDSTQAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYWMHWVKQRPGRGLEWIGRIDPNSGGTRYNEKFKSKATLTVDKPSSTAYMQLSSLTSEDSAVYYCARGLYDGNWYFDVWGTGTTVTVSSESQSFPNVFPLVSCESPLSDKNLVAESKYGPPCPSCPPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELGSLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 23 - protein, 543 aa, synthetic construct
MLRLLLALNLFPSIQVTGGSSGKPIPNPLLGLDSTQAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLEGGGGSGGGGSGGGGSQVQLQQPGAELVKPGASVKLSCKASGYTFTSYWMHWVKQRPGRGLEWIGRIDPNSGGTRYNEKFKSKATLTVDKPSSTAYMQLSSLTSEDSAVYYCARGLYDGNWYFDVWGTGTTVTVSSESQSFPNVFPLVSCESPLSDKNLVAESKYGPPCPPCPFWALVVVAGVLFCYGLLVTVALCVIWTNSRRNRGGQSDYMNMTPRRPGLTRKPYQPYAPARDFAAYRPKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 24 - protein, 21 aa, synthetic construct
MLRLLLALNLFPSIQVTGGSS

SEQ ID NO: 25 - protein, 21 aa, synthetic construct
MALPVTALLLPLALLLHAARP

SEQ ID NO: 26 - protein, 12 aa, synthetic construct
ESKYGPPCPSCP

SEQ ID NO: 27 - protein, 12 aa, synthetic construct
ESKYGPPCPPCP

SEQ ID NO: 28 - protein, 53 aa, synthetic construct
AAAGTTTTPAPRPPTPAPTIASQPLSLRPEACRPAAGGAVHTRGLDFACDRRP

SEQ ID NO: 29 - protein, 36 aa, synthetic construct
IEFMYPPPYLDNERSNGTIIHIKEKHLCHTQSSPKL

SEQ ID NO: 30 - protein, 14 aa, synthetic construct
GKPIPNPLLGLDST